(19) United States
(12) Patent Application Publication
(10) Pub. No.: US 2026/0297543 A1
(43) Pub. Date: Oct. 1, 2026

(54) ENGINEERED TET ENZYMES AND USE IN EPIGENETICS AND NEXT GENERATION SEQUENCING (NGS), SUCH AS TET-ASSISTED PYRIDINE BORANE SEQUENCING (TAPS)

(71) Applicant: Exact Sciences Innovation LTD., Oxford (GB)

(72) Inventors: Nicola A. Burgess-Brown, Oxford (GB); John Hinks, Oxford (GB); Marcello Tortorici, Oxford (GB); Rosa D'Agostino, Oxford (GB); Alejandra Fernandez-Cid, Oxford (GB)

(21) Appl. No.: 19/477,917

(22) PCT Filed: Apr. 24, 2024
(86) PCT No.: PCT/IB2024/054026

§ 371 Date: Oct. 23, 2025

Related U.S. Application Data
(60) Provisional application No. 63/498,874, filed on Apr. 28, 2023.

(51) Int. Cl. C12N 9/02; C12N 5/07; C12N 15/86; C12P 19/34; C12Q 1/6806; C12Q 1/6869

(52) U.S. Cl. CPC C12N 9/0071; C12N 5/0601; C12N 15/86; C12P 19/34; C12Q 1/6806; C12Q 1/6869; C07K 2319/00; C12N 2710/14043; C12Y 114/11

(57) ABSTRACT

The present invention relates to engineered TET enzymes that find use in epigenetics and Next Generation Sequencing (NGS), and more specifically to sequencing methods such as Tet-Assisted Pyridine Borane Sequencing (TAPS).

### STATEMENT OF RELATED CASES

This application claims the benefit of U.S. Provisional Appl. 63/498,874, filed Apr. 28, 2023, the contents of which are herein incorporated by reference in their entirety.

### SEQUENCE LISTING

The text of the computer readable sequence listing filed herewith, titled “41744-601_SEQUENCE_LISTING”, created Apr. 24, 2024, having a file size of 59,135 bytes, is hereby incorporated by reference in its entirety.

### FIELD OF THE INVENTION

The present invention relates to engineered TET enzymes that find use in epigenetics and Next Generation Sequencing (NGS), and more specifically to sequencing methods such as Tet-Assisted Pyridine Borane Sequencing (TAPS).

### BACKGROUND OF THE INVENTION

Cells in multicellular organisms are genetically homogeneous but structurally and functionally heterogeneous owing to the differential expression of genes. Epigenetics is the study of reversible, heritable changes in gene expression that do not involve changes to the underlying DNA sequence. Epigenetic mechanisms are mediated, among other means, by chemical modifications of the DNA such as by DNA methylation. In mammals, epigenetic regulation is crucial for a variety of different processes such as development, cell differentiation, and proliferation. A thorough understanding of the regulatory networks and epigenetic mechanisms that underlie context-specific gene expression programs and cellular phenotypes remains a critical scientific goal with broad implications for human health.

The need for methods able to determine the methylation state of DNA sequences as described above was until recently met by the combination of Next Generation Sequencing (NGS) with bisulfite sequencing (BS). The invention of Tet-Assisted Pyridine Borane Sequencing (TAPS) overcomes the many limitations of BS type techniques, particularly with respect to DNA sample damage and sensitivity. Sensitivity is of critical importance for the development of healthcare applications, such as Cancer Diagnostics, where sample DNA titers are low. A particular example would be the detection of cell free DNA (cfDNA) in blood.

Certain TAPS protocols utilize Ten-Eleven Translocase (TET) enzymes. The TET enzyme family oxidizes 5-methylcytosine (5mC) promoting locus-specific reversal of DNA methylation. Methylation of Cytosines is a frequent epigenetic modification in eukaryotes; 5-methylcytosine (5mC) is produced by DNA methyltransferase (Dnmt) activity and is located on CG dinucleotides (CpGs) of chromosomal DNA. TET proteins oxidize 5mC to 5-hydroxymethylcytosine (5hmC), 5-formylcytosine (5fmC), and 5-carboxylcytosine (5caC) to modify DNA methylation patterns.

The reliance of techniques involving TET for the enzymatic conversion of methylated DNA imbues the TET enzyme with considerable commercial and academic importance.

What is needed in the art are improved engineered versions of TET enzymes that are amenable to high-volume, low-cost production and that also exhibit the high performing functional characteristics essential for extremely sensitive methylation assay applications.

### SUMMARY OF THE INVENTION

The present invention relates to engineered TET enzymes that find use in epigenetics and Next Generation Sequencing (NGS), and more specifically to sequencing methods such as Tet-Assisted Pyridine Borane Sequencing (TAPS).

Accordingly, in some preferred embodiments, the present invention provides an engineered Ten-Eleven Translocase (TET) enzyme comprising a TET catalytic domain comprising at least one substitution mutation selected from the group consisting of: substitution of a non-cysteine amino acid in the catalytic domain with a cysteine; substitution of cysteine in the catalytic domain with a non-cysteine amino acid; and combinations thereof; wherein the engineered TET enzyme catalyzes the oxidation of 5-methylcytosine (5mC) to 5-hydroxymethylcytosine (5hmC) and/or 5hmC to 5-formylcytosine (5fC) and/or 5-carboxycytosine (5caC).

In some preferred embodiments, the at least one non-cysteine amino acid that is substituted with a cysteine occurs at a position in the catalytic domain of the TET enzyme that is occupied by a cysteine in an isoform or ortholog of the TET enzyme.

In some preferred embodiments, the TET catalytic domain is a mouse TET2 catalytic domain and the isoform is mouse TET1. In some preferred embodiments, the at least one non-cysteine amino acid that is substituted with a cysteine is at a position selected from the group consisting of 1286, 1291, 1322, 1835, and 1837 and combinations thereof, wherein the position numbers are based on position numbers for full length mouse TET2. In some preferred embodiments, the substitutions are selected from the group consisting of A1286C, S1291C, K1332C, M1835C and E1837C substitutions and combinations thereof, wherein the position numbers are based on position numbers for full length mouse TET2.

In some preferred embodiments, the at least one cysteine in the catalytic domain that is substituted with a non-cysteine amino acid occurs at a position in the catalytic domain of the TET enzyme that is occupied by a non-cysteine in an isoform of the TET enzyme.

In some preferred embodiments, the TET catalytic domain is a mouse TET2 catalytic domain and the isoform is a mouse TET1 enzyme. In some preferred embodiments, the at least one cysteine that is substituted with a non-cysteine amino acid is at a position selected from the group consisting of 1168, 1171, 1185, 1272, 1772, 1792 and 1827 and combinations thereof, wherein the position numbers are based on position numbers for full length mouse TET2. In some preferred embodiments, the substitutions are selected from the group consisting of C1168Y, C1171P, C1185T, C1272R, C1772S, C1792K and C1827G substitutions and combinations thereof, wherein the position numbers are based on position numbers for full length mouse TET2.

In some preferred embodiments, the catalytic domain of the engineered TET enzyme has an N terminal portion that shares at least 80% sequence identity with SEQ ID NO: 1 and a C terminal portion that shares at least 80% sequence identity to SEQ ID NO:2, with the proviso that the catalytic domain consists of at least one substitution selected from the group consisting of A1286C, S1291C, K1332C, M1835C, E1837C, C1168Y, C1171P, C1185T, C1272R, C1772S, C1792K and C1827G substitutions and combinations thereof, wherein the position numbers are based on position numbers for full length mouse TET2. In some preferred embodiments, the N terminal portion of the catalytic domain corresponds to SEQ ID NO: 1 and the C terminal portion of the catalytic domain corresponds to SEQ ID NO: 2. In some preferred embodiments, the N terminal portion of the catalytic domain and the C terminal portion of the catalytic domain are connected by a heterologous linker sequence.

In some preferred embodiments, the TET catalytic domain is a mouse TET3 catalytic domain and the isoform is mouse TET1. In some preferred embodiments, the at least one non-cysteine amino acid that is substituted with a cysteine is at a position selected from the group consisting of 1076, 1112, 1721, and 1726 and combinations thereof, wherein the position numbers are based on position numbers for full length mouse TET3. In some preferred embodiments, the substitutions are selected from the group consisting of A1076C, I1112C, M1721C, and E1726C substitutions and combinations thereof, wherein the position numbers are based on position numbers for full length mouse TET3. In some preferred embodiments, the engineered TET enzyme further comprises an amino acid substitution at a position selected from the group consisting of 975, 1658, and 1678 and combinations thereof, wherein the position numbers are based on position numbers for full length mouse TET3. In some preferred embodiments, the substitutions are selected from the group consisting of A975T, N1658S, and R1678K substitutions and combinations thereof, wherein the position numbers are based on position numbers for full length mouse TET3. In some preferred embodiments, the catalytic domain of the engineered TET enzyme has an N terminal portion that shares at least 80% sequence identity with SEQ ID NO:3 and a C terminal portion that shares at least 80% sequence identity to SEQ ID NO:4, with the proviso that the catalytic domain consists of at least 1 substitution selected from the group consisting of A1076C, I1112C, M1721C, E1726C, A975T, N1658S, and R1678K substitutions and combinations thereof, wherein the position numbers are based on position numbers for full length mouse TET3. In some preferred embodiments, the N terminal portion of the catalytic domain corresponds to SEQ ID NO:3 and the C terminal portion of the catalytic domain corresponds to SEQ ID NO:4. In some preferred embodiments, the N terminal portion of the catalytic domain and the C terminal portion of the catalytic domain are connected by a heterologous linker sequence.

In some preferred embodiments, the TET catalytic domain is a Lygus hesperus TET2 catalytic domain and the ortholog is mouse TET1. In some preferred embodiments, the at least one non-cysteine amino acid that is substituted with a cysteine is at a position selected from the group consisting of 740, 773, and 1400 and combinations thereof, wherein the position numbers are based on position numbers for partial Lygus hesperus TET2. In some preferred embodiments, the substitutions are selected from the group consisting of A740C, K773C, and Q1400C substitutions and combinations thereof, wherein the position numbers are based on position numbers for partial length Lygus hesperus TET2. In some preferred embodiments, the engineered TET enzyme further comprises an amino acid substitution at a position selected from the group consisting of 627, 641, 1337, and 1357 and combinations thereof, wherein the position numbers are based on position numbers for partial length Lygus hesperus TET2. In some preferred embodiments, the substitutions are selected from the group consisting of V627P, A641T, N1337S, and H1357K substitutions and combinations thereof, wherein the position numbers are based on position numbers for full length Lygus hesperus TET2. In some preferred embodiments, the catalytic domain of the engineered TET enzyme has an N terminal portion that shares at least 80% sequence identity with SEQ ID NO:5 and a C terminal portion that shares at least 80% sequence identity to SEQ ID NO:6, with the proviso that the catalytic domain consists of at least 1 substitution selected from the group consisting of A740C, K773C, Q1400C, V627P, A641T, N1337S, and H1357K, substitutions and combinations thereof, wherein the position numbers are based on position numbers for partial length Lygus hesperus TET2. In some preferred embodiments, the N terminal portion of the catalytic domain corresponds to SEQ ID NO:5 and the C terminal portion of the catalytic domain corresponds to SEQ ID NO:6. In some preferred embodiments, the N terminal portion of the catalytic domain and the C terminal portion of the catalytic domain are connected by a heterologous linker sequence.

In some preferred embodiments, the TET catalytic domain is a human TET2 catalytic domain and the ortholog is mouse TET1. In some preferred embodiments, the at least one non-cysteine amino acid that is substituted with a cysteine is at a position selected from the group consisting of 1373, 1409, 1921, and 1923 and combinations thereof, wherein the position numbers are based on position numbers for full length human TET2. In some preferred embodiments, the substitutions are selected from the group consisting of A1373C, K1409C, M1921C, and E1923C substitutions and combinations thereof, wherein the position numbers are based on position numbers for full length human TET2. In some preferred embodiments, the engineered TET enzyme further comprises an amino acid substitution at a position selected from the group consisting of 1258, 1272, 1858, and 1878 and combinations thereof, wherein the position numbers are based on position numbers for full length human TET2. In some preferred embodiments, the substitutions are selected from the group consisting of L1258P, A1272T, D1858S, and R1878K substitutions and combinations thereof, wherein the position numbers are based on position numbers for full length human TET2. In some preferred embodiments, the catalytic domain of the engineered TET enzyme has an N terminal portion that shares at least 80% sequence identity with SEQ ID NO:7 and a C terminal portion that shares at least 80% sequence identity to SEQ ID NO: 8 with the proviso that the catalytic domain consists of at least 1 substitution selected from the group consisting of A1373C, K1409C, M1921C, E1923C, L1258P, A1272T, D1858S, and R1878K substitutions and combinations thereof, wherein the position numbers are based on position numbers for full length human TET2 and the engineered TET enzyme catalyzes the oxidation of 5-methylcytosine. In some preferred embodiments, the N terminal portion of the catalytic domain corresponds to SEQ ID NO: 7 and the C terminal portion of the catalytic domain corresponds to SEQ ID NO:8. In some preferred embodiments, the N terminal portion of the catalytic domain and the C terminal portion of the catalytic domain are connected by a heterologous linker sequence.

In some preferred embodiments, the engineered TET enzymes have an N terminal truncation. In some preferred embodiments, the engineered TET enzymes have an N terminal truncation extending to the N-terminus of the catalytic domain.

In some preferred embodiments, the engineered TET enzymes have a C terminal truncation. In some preferred embodiments, the C terminal truncation is up to and including 77 amino acids. In some preferred embodiments, the C terminal truncation is up to and including 64 amino acids. In some preferred embodiments, the C terminal truncation is up to and including 44 amino acids.

In some preferred embodiments, all or a portion of the Low Complexity Region (LCR) of the engineered TET enzyme is deleted. In some preferred embodiments, the LCR is substituted with a heterologous linker sequence.

In some preferred embodiments, the engineered TET enzymes comprise at least one purification tag selected from the group consisting of a his tag and a FLAG tag and combinations thereof.

In some preferred embodiments, the present invention provides a nucleic acid encoding an engineered TET enzyme as described above. In some preferred embodiments, the nucleic acid further comprises a promoter in operable association with the nucleic acid encoding the engineered TET enzyme. In some preferred embodiments, the present invention provides a vector comprising the nucleic acid. In some preferred embodiments, the present invention provides a host cell comprising the nucleic acid or vector. In some preferred embodiments, the host cell is a prokaryotic host cell. In some preferred embodiments, the host cell is a eukaryotic host cell.

In some preferred embodiments, the present invention provides methods of producing an engineered TET enzyme comprising: culturing the host cell as described above under conditions such that the engineered TET enzyme is expressed; and isolating the expressed engineered TET enzyme.

In some preferred embodiments, the present invention provides methods of producing an engineered TET enzyme comprising: introducing a baculovirus vector comprising a nucleic acid sequence encoding the engineered TET enzyme into an insect cell free expression system under conditions such that the engineered TET enzyme is expressed; and isolating the expressed engineered TET enzyme.

In some preferred embodiments, the present invention provides methods of producing a recombinant TET enzyme comprising: introducing a baculovirus vector comprising a nucleic acid sequence encoding a TET enzyme into insect cell expression system under conditions such that the recombinant TET enzyme is expressed; and isolating the expressed recombinant TET enzyme. In some preferred embodiments, the recombinant TET enzyme is a recombinant mTET1, mTET2, or mTET3 enzyme or an engineered TET enzyme or ortholog as described above. In some preferred embodiments, the recombinant TET enzyme does not comprise an LCR. In some preferred embodiments, the LCR is substituted with a heterologous linker sequence. In some preferred embodiments, the recombinant TET enzyme has an N-terminal truncation. In some preferred embodiments, the N-terminal truncation extends to the N terminus of the catalytic domain of the recombinant TET enzyme.

In some preferred embodiments, the present invention provides a recombinant TET enzyme produced by the foregoing methods.

In some preferred embodiments, the present invention provides recombinant TET enzymes comprising a catalytic domain having at least 80%, 90%, 95%, 98% or 100% identity to the catalytic domain of a sequence selected from the group consisting of SEQ ID NOs: 19 to 33 wherein the recombinant TET enzyme catalyzes the oxidation of 5-methylcytosine (5mC) to 5-hydroxymethylcytosine (5hmC) and/or 5hmC to 5-formylcytosine (5fC) and/or 5-carboxycytosine (5caC). In some preferred embodiments, the enzyme is modified by deletion of the TET LCR. In some preferred embodiments, the LCR is substituted with a heterologous linker sequence. In some preferred embodiments, the recombinant TET enzyme has an N-terminal truncation. In some preferred embodiments, the N-terminal truncation extends to the N terminus of the catalytic domain of the recombinant TET enzyme. In some preferred embodiments, the enzyme comprises at least one purification tag selected from the group consisting of a his tag and a FLAG tag and combinations thereof. In some preferred embodiments, the present invention provides a nucleic acid encoding the recombinant TET enzyme. In some preferred embodiments, the nucleic acid further comprises a promoter in operable association with the nucleic acid encoding the recombinant TET enzyme. In some preferred embodiments, the present invention provides a vector comprising the nucleic acid. In some preferred embodiments, the present invention provides a host cell comprising the nucleic acid or vector. In some preferred embodiments, the host cell is a prokaryotic host cell. In some preferred embodiments, the host cell is a eukaryotic host cell.

In some preferred embodiments, the present invention provides methods of modifying a nucleic acid in a nucleic acid sample comprising 5mC and/or 5hmC comprising: contacting the nucleic acid with an engineered or recombinant TET enzyme as described above so that 5mC and/or 5hmC is converted 5fC and/or 5caC to provide an oxidized target nucleic acid. In some preferred embodiments, the TET enzyme is an engineered TET enzyme as described above.

In some preferred embodiments, the methods further comprise the step of contacting the oxidized target nucleic acid with a reducing agent. In some preferred embodiments, the reducing agent is a borane reducing agent and the 5fC and/or 5caC is reduced to dihydrouracil (DHU). In some preferred embodiments, the borane reducing agent comprises an agent selected from the group consisting of 2-picoline borane (pic-BH3), borane, sodium borohydride, sodium cyanoborohydride, and sodium triacetoxyborohydride.

In some preferred embodiments, the methods further comprise contacting the oxidized target nucleic acid with sodium bisulfite so that the 5fC and/or 5caC is deaminated to uracil (U).

In some preferred embodiments, the methods further comprise contacting the oxidized target nucleic acid with an APOBEC (Apolipoprotein B mRNA Editing Catalytic Polypeptide-like) enzyme so that the 5fC and/or 5caC is deaminated to uracil (U).

In some preferred embodiments, the methods further comprise adding a blocking group to one or more of the 5mC and/or 5hmC in the nucleic acid sample. In some preferred embodiments, the blocking group is added prior to contacting with the oxidizing agent. In some preferred embodiments, 5hmC is blocked. In some preferred embodiments, the blocking group comprises a sugar or a uridine diphosphate (UDP)-linked sugar. In some preferred embodiments, the blocking group is added after contacting with the oxidizing agent and prior to contacting with the borane reducing agent. In some preferred embodiments, the one or more modified cytosines comprises 5caC or 5fC. In some preferred embodiments, the blocking group comprises an aldehyde reactive compound. In some preferred embodiments, the aldehyde reactive compound comprises a hydroxylamine derivative, a hydrazine derivative, or a hydrazide derivative. In some preferred embodiments, adding the blocking group comprises contacting the nucleic acid sample with (i) a coupling agent and (ii) an amine, hydrazine, or hydroxylamine compound.

In some preferred embodiments, the methods further comprise sequencing the nucleic acid after contacting with the borane reducing agent to identify converted cytosine bases.

In some preferred embodiments, the present invention provides a system or kit for oxidizing a methylated nucleotide comprising a TET enzyme (e.g., engineered, or recombinant TET enzyme) as described above.

In some preferred embodiments, the systems or kits further comprise a borane reducing agent. In some preferred embodiments, the borane reducing agent comprises an agent selected from the group consisting of 2-picoline borane (pic-BH3), borane, sodium borohydride, sodium cyanoborohydride, and sodium triacetoxyborohydride.

In some preferred embodiments, the systems or kits further comprise sodium bisulfite.

In some preferred embodiments, the systems or kits further comprise an APOBEC (Apolipoprotein B mRNA Editing Catalytic Polypeptide-like) enzyme.

In some preferred embodiments, the systems or kits further comprise a blocking reagent. In some preferred embodiments, the blocking reagent is selected from the group consisting of a sugar or a uridine diphosphate (UDP)-linked sugar and an aldehyde reactive compound. In some preferred embodiments, the aldehyde reactive compound is selected from the group consisting of a hydroxylamine derivative, a hydrazine derivative, and a hydrazide derivative. In some preferred embodiments, the blocking reagent is a sugar or a uridine diphosphate (UDP)-linked sugar and the system or kit further comprises a glucosyltransferase enzyme.

### BRIEF DESCRIPTION OF THE DRAWINGS

FIG. 1. Schematic diagram of Mouse TET (also referred to herein as MmTET or mTET) enzyme domain organization, showing the catalytic domain of mouse TET isoforms including an N terminal portion including Cys-N, Cys-C and a conserved double-stranded β-helix (DSBH) sub-domain bisected by a Low Complexity Region (LCR).

FIG. 2. Typical Size Exclusion Chromatography (SEC) profile for Nickel (Ni) IMAC purified E. coli expressed MmTET1 Full Length (FL) Catalytic Domain (CD).

FIG. 3. LCR removal and linker insertion strategies.

FIG. 4. Alignment of the HsTET2 CD sequence from the published Protein Data Bank (PDB 5D9Y) with MmTET1 CD and MmTET2 CD locations of various C-terminus truncations.

FIG. 5. Summary of domain exchange and construct design impacts tested. This figure illustrates the results from 80 TET enzyme CD constructs expressed at 50 ml scale in E. coli, purified by affinity chromatography, then assessed for purity by SDS-PAGE and for activity via an in-house assay. The following terms are used in the figure. Arrows=domain swaps between MmTET1 and MmTET2. C-term tagging=replacing the N-terminal tags with equivalent C-terminal tags. GGS=glycine, glycine, serine addition ahead of the first native CD residue. Minor Loop=an unstructured region reported in Rat and some depositions of MmTET1 isoforms only. N-term extension=inclusion of up to 20 amino acids from the TET1 sequence upstream of the Cys-N domain start.

FIG. 6. Detailed scheme of MmTET2 features relating to CD engineering.

FIG. 7A-B. The cysteine swaps as applied to MmTET2 improved enzyme activity and developability. (A) SDS-PAGE analysis of MmTET2 purified from insect cells. Aggregation is greater in MmTET2 deltaLCR than in MmTET2 deltaLCR Cys swap. (B) Summary table with yield and % conversion when used in TAPS.

FIG. 8A-B. Comparison of SDS-PAGE analysis between MmTET2 deltaLCR vs MmTET2 N-His-Flag CD deltaLCR 1xGS CysSwap-44 C-term (referred to herein as TET v1.0 and represented by SEQ ID NO: 16). (A) SDS-PAGE analysis of MmTET2 deltaLCR purified from E. coli (doublet band). (B) SDS-PAGE analysis of TET v1.0 purified from E. coli (resolved band).

FIG. 9. Comparison of TAPS performance (represented by conversion %) between MmTET2 CD deltaLCR and TET v1.0 at 30 and 60 minutes, showing the difference between MmTET2 deltaLCR with Cys swap (TET v1.0) and without Cys swap (MmTET2 CD deltaLCR).

FIG. 10. Comparison of commercial MmTET2 to TET v1.0 by relative performance in TAPS.

FIG. 11. Relative activity of MmTET2 ALCR vs TET v1.0 over (A) a range of temperatures, (B) a range of pH, and (C) a range of NaCl concentrations.

FIG. 12. SPR sensorgram comparison of TET v1.0 with commercial MmTET2.

FIG. 13. Percent conversion of fully methylated Lambda generated from sequencing data of either TAPS converted or EM-seq converted DNA.

### DETAILED DESCRIPTION OF THE INVENTION

The present invention relates to engineered TET enzymes that find use in epigenetics and Next Generation Sequencing (NGS), and more specifically to sequencing methods such as Tet-Assisted Pyridine Borane Sequencing (TAPS).

TET enzymes occur in nature across the eukaryotes (predominantly), with mammals having three isoforms (other organisms have varying numbers of isoforms), each exhibiting subtle differences in cellular distribution, activity and sequence. Methylation state sequencing techniques such as TAPS can utilize a TET enzyme to convert methylated DNA into any of its various oxidation states (5hmC, 5fmC and 5caC) which may subsequently either be read directly (e.g., by third generation, or “Long Read” NGS) or chemically converted into alternate bases such as Uracil to be read by conventional NGS (e.g., by second generation, or “Short Read” NGS) techniques.

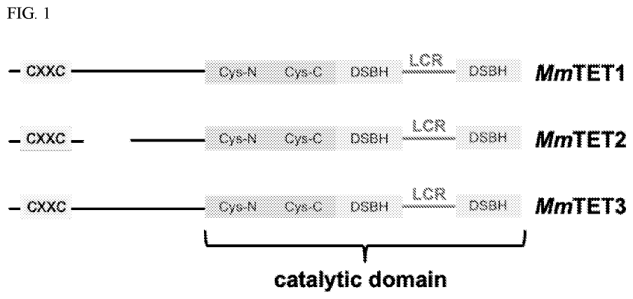

A number of TET enzymes are known in the art. Wild-type TET enzymes are large and comprise extensive regions of negligible structural integrity, making them prone to aggregation, degradation and low yields when expressed heterogeneously (e.g., for manufacturing purposes). A Catalytic Domain (CD) at their C-terminal end comprises a conserved double-stranded β-helix (DSBH) sub-domain bisected by a Low Complexity Region (LCR), a cysteine-rich region, and cofactor binding sites for Fe (II) and 2-oxoglutarate (2-OG). Together these form the core catalytic region of the enzyme as illustrated for the mouse TET isoforms in FIG. 1.

Native, functionally active TET enzymes are not ideal for high volume manufacture. In particular, these TET enzymes are typically low yield and prone to aggregation and degradation. Furthermore, TET enzymes that have so far been engineered to display improved manufacturability do not possess the requisite functional characteristics for use in stringent, high sensitivity assay formats (e.g., NGS applications within healthcare and specifically TAPS assays).

The present invention addresses this problem by providing engineered TET enzymes with improved manufacturing characteristics and performance in methylation assays such as TAPS. In preferred embodiments, the engineered TET enzymes comprise one or more amino acid substitutions in the catalytic domain so that cysteine distribution of one TET isoform (e.g., mouse TET1 or human TET1) is replicated in a different TET isoform or ortholog (e.g., mouse TET2, human TET2, mouse TET3, or Lygus hesperus TET2). In order to replicate the cysteine distribution, the substitutions can include 1) substitution of one or more non-cysteine amino acids in the catalytic domain of the target TET enzyme (e.g., mouse TET2, human TET2, mouse TET3 or Lygus hesperus TET2) with a cysteine at a position that is occupied by a cysteine in the catalytic domain of a TET isoform or ortholog (e.g., mouse TET1 or human TET1); and/or 2) substitution of one or more cysteines in the catalytic domain of the target TET enzyme (e.g., mouse TET2, human TET2, mouse TET3, or Lygus hesperus TET2) with a non-cysteine amino acid at a position that is occupied by a non-cysteine amino acid in the catalytic domain of a TET isoform or ortholog (e.g., mouse TET1 or human TET1). Surprisingly, engineered TET enzymes with these substitutions demonstrate improved solubility and stability with reduced aggregation as compared to non-mutated versions without compromising yield in both insect or bacterial expression systems. Furthermore, the engineered TET enzymes display improved performance in TAPS assays as compared to the wild-type enzymes.

It is highly desirable to develop TET enzymes to meet the stringent requirements of TAPS assays, which is superior to other methods for mapping DNA modifications (including 5mC and 5hmC). These other methods, for instance bisulfite-based methods, include methods that incorporate enzymatic modifications by TET alongside bisulfite treatment (such as TAB-seq), and more recent methods that avoid bisulfite use (such as EM-seq), as these methods cannot provide full sequence and epigenetic information from a single sequencing operation, whereas TAPS can. More specifically, because TAPS provides greater sensitivity than other techniques, an enzyme able to provide maximum conversion rates and cause the least DNA loss is necessary if the full potential of TAPS is to be realized. Furthermore, that enzyme must be capable of commercial production and be robust enough to incorporate into kits, while retaining its high conversion rate activity. Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” or “consisting essentially of” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “TET enzyme” refers to a member of the Ten-Eleven Translocation (TET) family that oxidizes 5-methyl cytosine (5mC) to 5-hydroxymethylcytosine (5hmC) and 5hmC to 5-carboxylcytosine (5caC) and 5-formylcytosine (5fC).

As used herein, the term “engineered TET enzyme” refers to a TET enzyme that has one or more changes in its amino acid sequence as compared to the wild-type version of the TET enzyme. The changes in amino acid sequence may be one or more of an amino acid substitution, amino acid deletion, truncation of the TET enzyme, or addition of amino acids or functional sequences to the TET enzyme.

The percentage of identity of an amino acid sequence or nucleic acid sequence, or the term “% sequence identity”, is defined herein as the percentage of residues of the full length of an amino acid sequence or nucleic acid sequence that is identical with the residues in a reference amino acid sequence or nucleic acid sequence after aligning the two sequences and introducing gaps, if necessary, to achieve the maximum percent identity. The percentage homology of an amino acid sequence or the term “% homology to” is defined herein as the percentage of amino acid residues in a particular sequence that are homologous with the amino acid residues in a reference sequence, after aligning the sequences and introducing gaps, if necessary, to achieve the maximum percent sequence homology. This method takes into account conservative amino acid substitutions. Conservative substitutions are substitutions of an amino acid to be substituted with a similar amino acid. Amino acids can be similar in several characteristics, for example, size, shape, hydrophobicity, hydrophilicity, charge, isoelectric point, polarity, aromaticity, etc. Preferably, a conservative substitution is an exchange of one amino acid within a group for another amino acid within the same group, whereby the groups are the following: (1) alanine, valine, leucine, isoleucine, methionine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

The terms “protein” and “polypeptide” refer to compounds comprising amino acids joined via peptide bonds and are used interchangeably. A protein or polypeptide encoded by a gene is not limited to the amino acid sequence encoded by a gene, but may include post-translational modifications of one or more amino acids of the protein or polypeptide. Sequences of proteins and polypeptides are depicted herein from N-terminal to C-terminal, unless otherwise indicated. As used herein with respect to the amino acids sequence of a protein or polypeptide, the terms “N-terminal” and “C-terminal” refer to relative positions in the amino acid sequence of the protein or polypeptide toward the N-terminus and the C-terminus, respectively. “N-terminus” and “C-terminus” refer to the extreme amino and carboxyl ends of the polypeptide, respectively.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide.

As used herein, a “methylated nucleic acid molecule” refers to a nucleic acid molecule that contains one or more methylated nucleotides.

As used herein, a “methylation state”, “methylation profile”, “methylation status,” or “methylation signature” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, “methylation frequency” or “methylation percent (%)” refer to the number of instances in which a molecule or locus is methylated relative to the number of instances the molecule or locus is unmethylated. Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, the present technology provides for the diagnosis of mammals such as humans, as well as those mammals of importance due to being endangered; of economic importance, such as animals raised on farms for consumption by humans; and/or of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses.

As used herein the term “nucleic acid sample” refers to nucleic acid obtained from an organism from the Monera (bacteria), Protista, Fungi, Plantae, and Animalia Kingdoms. The nucleic acid may also be obtained from a virus. Nucleic acid samples may be obtained from a patient or subject, from an environmental sample, or from an organism of interest (e.g., both cellular and circulating cell-free DNA (cfDNA) obtained from tissue, a cell, collection of cells, blood, plasma, serum, organ secretion, semen (seminal fluid), vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, or any other bodily fluid comprising a desired nucleic acid or cfDNA), DNA obtained from biopsies, and DNA obtained from cells, secretions, or tissues from the lymph gland, breast, liver, bile ducts, pancreas, mouth, stomach, colon, rectum, esophagus, small intestine, appendix, duodenum, polyps, gall bladder, anus, prostate, endometrium, vagina, ovary, cervix, skin, bladder, kidney, lung, and/or peritoneum. In other embodiments, the target nucleic acid may be obtained from a sample that contains diseased tissue or cells, or is suspected of containing diseased tissue or cells (e.g., a sample that is cancerous, or contains cancerous tissue or cells, or is suspected of being cancerous or suspected of containing cancerous tissue or cells). In some embodiments, the nucleic acid sample is obtained from a subject that has a disease or disorder (e.g., cancer), is suspected of having the disease or disorder, or is being screened to determine the presence of the disease or disorder. In some embodiments, the nucleic acid sample is circulating cell-free DNA (cell-free DNA or cfDNA), for instance DNA found in the blood and is not present within a cell. As would be recognized by one of ordinary skill in the art based on the present disclosure, cfDNA can be isolated from a bodily fluid using methods known in the art. Commercial kits are available for isolation of cfDNA including, for example, the Circulating Nucleic Acid Kit (Qiagen). The nucleic acid sample may result from an enrichment step, including, but not limited to antibody immunoprecipitation, chromatin immunoprecipitation, restriction enzyme digestion-based enrichment, hybridization-based enrichment, or chemical labeling-based enrichment.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Engineered TET Enzymes

In some preferred embodiments, the present invention provides engineered TET enzymes comprising a TET catalytic domain comprising one or more changes to at least one amino acid. In some preferred embodiments, the changes to at least one amino acid may comprise, consist essentially of, or consist of a substitution, deletion, addition, or truncation. In some preferred embodiments, the changes to at least one amino acid may comprise, consist essentially of, or consist of one or more amino acid substitutions. In some preferred embodiments, the substitutions comprise, consist essentially of, or consist of 1) substitution of one or more non-cysteine amino acids in the catalytic domain of the engineered TET enzyme with a cysteine; and/or 2) substitution of cysteine in the catalytic domain of the engineered TET enzyme with a non-cysteine amino acid. In some preferred embodiments, substitution of the one or more non-cysteine amino acids in the catalytic domain of the engineered TET enzyme with a cysteine is at a position that is occupied by a cysteine in the catalytic domain of a TET isoform or ortholog of the engineered TET enzyme. In some preferred embodiments, substitution of the one or more cysteines in the catalytic domain of the target TET enzyme with a non-cysteine amino acid is at a position that is occupied by a non-cysteine amino acid in the catalytic domain of a TET isoform or ortholog of the engineered TET enzyme. By making these substitutions or “cysteine swaps,” the cysteine distribution in the catalytic domain of the engineered TET enzyme is made to replicate or more closely replicate the cysteine distribution in an isoform or ortholog of the engineered TET enzyme. Based on the guidance herein, the cysteine swaps may be performed between any orthologs from any species. For example, the swaps may be performed between TET1, TET2, or TET3 orthologs from the same or different species. Exemplary species include, but are not limited to, Mus musculus, Homo sapiens, Naegleria gruberi, Coprinopsis cinerea Lygus hesperus, Drosophila melanogaster, Xenopus laevis, Aedes aegipty, Anopheles gambia, Drosophila ananassae, Cavia porcellus, Lama pacos, Macaca fascicularis, Mus pahari, Oryctolagus cuniculus, Chanos chanos, Sus scrofa, Xenopus tropicalis, etc. In some particularly preferred embodiments, the engineered TET enzyme may be mouse TET2, human TET2, mouse TET3 or Lygus hesperus TET2 and the isoform or ortholog of the engineered TET enzyme may be mouse TET1 or human TET1.

The present invention is not limited to engineered TET enzymes from any particular species (e.g., the exemplary species listed in the preceding paragraph). In some particularly preferred embodiments, the engineered TET enzyme may be a mouse TET enzyme, a human TET enzyme, or a Lygus hesperus TET enzyme. Indeed, the cysteine substitution principles described herein can be applied to any TET enzyme with a known sequence. Likewise, the present invention is not limited to any particular TET isoform. Suitable TET isoforms include TET1, TET2 and TET3 isoforms. Likewise, the present invention is not limited to engineered TET enzymes with any particular number of catalytic domain amino substitutions. For example, the engineered TET enzymes may comprise from 1 to 12, or 1, 2, 3, 4, 5, 6, 7, 8, 9, 10, 11, 12 substitutions in the catalytic domain.

In some preferred embodiments, the engineered TET enzymes comprise one or more deletions, truncations or additions as compared to the corresponding wild-type enzymes. In some embodiments, the engineered TET enzyme is truncated at its N terminus as compared to the corresponding wild-type enzyme. In some preferred embodiments, the N terminal truncation extends to the N terminal portion of the catalytic domain of the TET enzyme. In some embodiments, the engineered TET enzyme is truncated at its C terminus as compared to the corresponding wild-type enzyme. In some embodiments, the C terminal truncation is from 10 to 77, 20 to 77, 30 to 77, 10 to 64, 20 to 64, 30 to 64, 10 to 44, 20 to 44, 30 to 44, 10 to 50, 20 to 50, 30 to 50, 10 to 55, 20 to 55, 30 to 55, 10 to 60, 20 to 60, 30 to 60, 10 to 70, 20 to 70, 30 to 70, 10 to 75, 20 to 74 or 30 to 75 amino acids as compared to the corresponding wild-type enzyme. In some embodiments, the C terminal truncation is about 44, 50, 55, 60, 64, 70, 75 or 77 amino acids as compared to the corresponding wild-type enzyme, wherein about is +/−five amino acids. In some embodiments, the C terminal truncation is 77 amino acids as compared to the corresponding wild-type enzyme. In some embodiments, the C terminal truncation is 64 amino acids as compared to the corresponding wild-type enzyme. In some embodiments, the C terminal truncation is 44 amino acids as compared to the corresponding wild-type enzyme.

In some preferred embodiments, all or a portion of the Low Complexity Region (LCR) in the catalytic domain of the engineered TET enzyme is deleted as compared to the wild-type enzyme. In some preferred embodiments, at least from 50, 100, 150, 200, 250, 300, 350, or 375 amino acids up to the full length of the LCR are deleted. In some preferred embodiments, about 375 amino acids of the LCR are deleted, where about is +/−5 amino acids. For example, with reference to amino acid numbering for the full length mouse TET2 sequence (SEQ ID NO:9), amino acids E1373 to A1754 or K1379 to A1745 may be deleted. In some preferred embodiments, the deleted LCR is replaced with a linker sequence. Any suitable linker sequence as is known in the art may be utilized, such as flexible glycine/serine linker sequences. In some preferred embodiments, the linker has or consists of the sequence GGGGSGGGGSGGGGS (SEQ ID NO:13).

The engineered TET enzymes may further be modified to include one or more sequences that facilitate purification of the engineered TET enzyme following its expression in a suitable expression system. In some preferred embodiments, the engineered TET enzyme comprises a His tag, such as a 6×His tag, preferably at the N terminus of the engineered TET enzyme. In some preferred embodiments, the engineered TET enzyme comprises a Flag tag (e.g., DYKDDDDK (SEQ ID NO:14)), preferably at the N terminus of the engineered TET enzyme. In some preferred embodiments, the engineered TET enzyme comprises both a His tag and Flag tag (e.g., SEQ ID NO:35), which are preferably located at the N terminus of the engineered enzyme. In some preferred embodiments, a GGS sequence is included between the tag(s) and the N terminal portion of the catalytic domain of the TET enzyme.

In some particularly preferred embodiments, the engineered TET enzyme comprises a mouse TET2 catalytic domain, mouse TET3 catalytic domain, human TET2 catalytic domain or Lygus hesperus TET2 catalytic domain with one or more substitutions, most preferably cysteine swaps, in the catalytic domain. Referring to FIG. 1, the catalytic domain of mouse TET isoforms includes an N terminal portion including Cys-N, Cys-C and DSBH regions and a C terminal portion including a DSBH region, separated by the LCR. As indicated above, all or a portion of the LCR is deleted and preferably replaced by a linker sequence in preferred embodiments of the invention. Likewise, in preferred embodiments the N-terminal and/or the C terminal is truncated as compared to the wild type enzyme. It will be appreciated that in these preferred embodiments, the engineered enzymes comprise an engineered catalytic domain wherein the N terminal and C terminal portions of the catalytic domain (with the one or more specified substitutions) are joined by a linker sequence.

In Table 1 below, preferred sequences of the N and C terminal portions of the catalytic domains for engineered mouse TET2, mouse TET3, human TET2 and Lygus hesperus TET2 are provided. In preferred embodiments, these sequences are linked by a linker sequence. The specific substitutions in these sequences are described in more detail below.

TABLE 1

| Engineered | | |
|---|---|---|
| TET | N terminal portion of catalytic | C terminal portion of catalytic |
| enzyme | domain | domain |
| Mouse | SEQ ID NO: 1 | SEQ ID NO: 2 |
| TET2 | QSQNGKCEGCNPDKDEAPYYTHLGAGPDV | VQEIEYWSDSEHNFQDPSI |
| | AAIRTLMEERYGEKGKAIRIEKVIYTGKEGK | GGVAIAPTHGSILIECAKKE |
| | SSQGCPIAKWVYRRSSEEEKLLCLVRVRPN | VHATTKVNDPDRNHPTRIS |
| | HTCETAVMVIAIMLWDGIPKLLASELYSELT | LVLYRHKNLFLPKHGLAL |
| | DILGKYGIPTNRRCSQNETRNCTCQGENPET | WEAKCACKARKEEECGK |
| | CGASFSFGCSWSMYYNGCKFARSKKPRKFR | NGSDHVSQKNHGKQEKRE |
| | LHGAEPKEEERLGSHLQNLATVIAPIYKKLA | PTG |
| | PDAYNNQVEFEHQAPDCRLGLKEGRPFSGV | |
| | TCCLDFCAHSHRDQQNMPNGSTVVVTLNR | |
| | EDNREVGACPEDEQFHVLPMYIIAPEDEFGS | |
| | TEGQEKKIRMGSIEVLQSFRRRRVIRIG | |
| Mouse | SEQ ID NO: 3 | SEQ ID NO: 4 |
| TET3 | EFPTCDCVEQIVEKDEGPYYTHLGSGPTVAS | EELWSDSEHNFLDESIGGV |
| | IRELMEDRYGEKGKAIRIEKVIYTGKEGKSS | AVAPAHCSILIECARKELH |
| | RGCPIAKWVIRRHTLEEKLLCLVRHRAGHH | ATTPLKKPNRCHPTRISLVF |
| | CQNAVIVILILAWEGIPRSLGDTLYQELTDTL | YQHKNLNQPNHGLALWE |
| | RKYGNPTSRRCGLNDDRTCTCQGKDPNTCG | AKCKQLACRARQRQEEAA |
| | ASFSFGCSWSMYFNGCKYARSKTPRKFRLT | RLGLGQQEAKLYGKKRK |
| | GDNPKEEEVLRNSFQDLATEVAPLYKRLAP | WGGAMVAEPQHKEKKGA |
| | QAYQNQVTNEDVAIDCRLGLKEGRPFSGVT | IPTRQALAMPTDSAVTVSS |
| | CCMDFCAHAHKDQHNLYNGCTVVCTLTKE | YAYTKVTGPYSRWI |
| | DNRCVGQCPEDEQLHVLPLYKMASTDEFGS | |
| | EENQNAKVSSGAIQVLTAFPREVRRLPEPAK | |
| | S | |
| Lygus | SEQ ID NO: 5 | SEQ ID NO: 6 |
| hesperus | EEDIKLSPEQIERLKANARGDVPGCECFPPD | VTDFTNNEDCFSDPSVGGV |
| TET2 | QLPPEPGPYYTHLGTAKSLEELRENVEKRTG | AIALSHGAVLFEVAKKELH |
| | LTGKAIRIEKVLFTGKEGKTTSGCPLGKWIIR | ATTALKIPNRFHPTRISLVF |
| | RSGPEEKVLMVVKHRKGHVCVNSWIVIVTV | YQHRNLNKSDHGRGEWK |
| | AWEGVPAAMADDLYATVTDKLKNYGVPTS | EKCRCKKELAEKHAEMAE |
| | RRCGVNEPRTCTCQGLDNTTCGASFSFGCS | MAERAAELAEKATSTASIA |
| | WSMYYNGCKFARSQTARKFKLSVKTEEQC | TQSLDVQTEPQPVGAIMD |
| | MEDKLQNLATYLSPVYKTMAPESFSNMTQ | HMPYNYYPMQQQPPSPLN |
| | HEQEAVDCRLGLQPGRPWSGVTCCFDFCAH | RYSPMVATSSIQSSPSPQM |
| | AHRDIHNMNNGCTTVVTLTKHRGFECPEDE | QPVSTPSAWGNLFPLHPSV |
| | QLHVLPLYVIDQSDEFGSKEGYHEKVRSGAI | VQGPYQESETLG |
| | EVLTKYPCEIRTRQAPLES | |
| Human | SEQ ID NO: 7 | SEQ ID NO: 8 |
| TET2 | EKDEGPFYTHLGAGPNVAAIREIMEERFGQ | DEVWSDSEQSFLDPSIGGV |
| | KGKAIRIERVIYTGKEGKSSQGCPIAKWVVR | AVAPTHGSILIECAKKELH |
| | RSSSEEKLLCLVRERAGHTCEAAVIVILILV | ATTPLKNPNRNHPTRISLV |
| | WEGIPLSLADKLYSELTETLRKYGTPTNRRC | FYQHKSMNEPKHGLALWE |
| | ALNEERTCTCQGLDPETCGASFSFGCSWSM | AKCACKAREKEEECEKYG |
| | YYNGCKFARSKIPRKFKLLGDDPKEEEKLES | PDYVPQKSHGKKVKREPA |
| | HLQNLSTLMAPTYKKLAPDAYNNQIEYEHR | EPHETSEPTYLRFIKSLAER |
| | APECRLGLKEGRPFSGVTCCLDFCAHAHRD | TMSVTTDSTVTTSPYAFTR |
| | LHNMQNGSTLVCTLTREDNREFGGCPEDEQ | VTGPYNRYI |
| | LHVLPLYKVSDVDEFGSVEAQEEKKRSGAI | |
| | QVLSSFRRKVRMLAEPVKT | |

In some particularly preferred embodiments, the engineered TET enzyme comprises a mouse TET2 catalytic domain comprising one or more substitutions, the one or more substitutions comprising, consisting essentially of, or consisting of one or more of the following substitutions: A1286C, S1291C, K1332C, M1835C, E1837C, C1168Y, C1171P, C1185T, C1272R, C1772S, C1792K and C1827G, wherein the positions are based on position numbering from full length mouse TET2 (SEQ ID NO:9). In some preferred embodiments, the one or more substitutions of the mouse TET2 catalytic domain comprise, consist essentially of, or consist of 1, 2, 3, 4, 5, 6, 7, 8, 9, 10, 11, or 12 of the substitutions in any combination. In some preferred embodiments, the catalytic domain of the engineered mouse TET2 enzyme has an N terminal portion that shares at least 90%, 95% or 98%, 99% or 100% sequence identity with SEQ ID NO:1 and a C terminal portion that shares at least 90%, 95%, 98%, 99%, or 100% sequence identity to SEQ ID NO:2, with the proviso that the catalytic domain comprises at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the catalytic domain of the engineered mouse TET2 enzyme has an N terminal portion that shares at least 90%, 95% or 98%, 99% or 100% sequence identity with SEQ ID NO: 1 and a C terminal portion that shares at least 90%, 95%, 98%, 99%, or 100% sequence identity to SEQ ID NO: 2, with the proviso that the catalytic domain comprises one or more substitutions consisting of at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the catalytic domain of the engineered mouse TET2 enzyme has an N terminal portion that has 100% sequence identity with SEQ ID NO: 1 and a C terminal portion that has 100% sequence identity to SEQ ID NO: 2.

In some preferred embodiments, the engineered mouse TET2 enzyme shares at least 90%, 95%, 98%, 99% or 100% identity with SEQ ID NO: 15, with the proviso that the catalytic domain comprises at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the engineered mouse TET2 enzyme shares at least 90%, 95%, 98%, 99% or 100% identity with SEQ ID NO:15, with the proviso that the catalytic domain comprises one or more substitutions consisting of at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the engineered mouse TET2 enzyme is set forth in SEQ ID NO:15. The engineered mouse TET2 enzyme of SEQ ID NO:15 has an N terminal truncation extending to the N terminal portion of the catalytic domain, a C terminal truncation of 44 amino acids, and the LCR is replaced with a GS linker sequence.

In some preferred embodiments, the engineered mouse TET2 comprises a polypeptide sequence having at least 90%, 95%, 98%, 99% or 100% identity with SEQ ID NO: 15, with the proviso that the catalytic domain comprises the following substitutions: A1286C, S1291C, K1332C, M1835C, E1837C, C1168Y, C1171P, C1185T, C1272R, C1772S, C1792K and C1827G. In some preferred embodiments, the engineered mouse TET2 comprises a polypeptide sequence having at least 90%, 95%, 98%, 99% or 100% identity with SEQ ID NO: 15, with the proviso that the catalytic domain comprises one or more substitutions consisting of the following substitutions: A1286C, S1291C, K1332C, M1835C, E1837C, C1168Y, C1171P, C1185T, C1272R, C1772S, C1792K and C1827G.

In some preferred embodiments, the engineered mouse TET2 enzyme shares at least 90%, 95%, 98%, 99% or 100% identity with SEQ ID NO:16, with the proviso that the catalytic domain comprises at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the engineered mouse TET2 enzyme shares at least 90%, 95%, 98%, 99% or 100% identity with SEQ ID NO: 16, with the proviso that the catalytic domain comprises one or more substitutions consisting of at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the engineered mouse TET2 enzyme is set forth in SEQ ID NO:16. The engineered mouse TET2 enzyme of SEQ ID NO: 16 has His and Flag tags at the N terminus, an N terminal truncation extending to the N terminal portion of the catalytic domain, a C terminal truncation of 44 amino acids, and the LCR is replaced with a GS linker sequence.

In some preferred embodiments, the engineered mouse TET2 comprises a polypeptide sequence having at least 90%, 95%, 98%, 99% or 100% identity with SEQ ID NO: 16, with the proviso that the catalytic domain comprises at least one substitution selected from the group consisting of A1286C, S1291C, K1332C, M1835C, E1837C, C1168Y, C1171P, C1185T, C1272R, C1772S, C1792K and C1827G and combinations thereof. In some preferred embodiments, the engineered mouse TET2 comprises a polypeptide sequence having at least 90%, 95%, 98%, 99% or 100% identity with SEQ ID NO:16, with the proviso that the catalytic domain comprises one or more substitutions consisting of at least one substitution selected from the group consisting of A1286C, S1291C, K1332C, M1835C, E1837C, C1168Y, C1171P, C1185T, C1272R, C1772S, C1792K and C1827G and combinations thereof.

In some preferred embodiments, the engineered mouse TET2 enzyme shares at least 90%, 95%, 98%, 99% or 100% identity with SEQ ID NO: 16, with the proviso that the catalytic domain comprises the following substitutions: A1286C, S1291C, K1332C, M1835C, E1837C, C1168Y, C1171P, C1185T, C1272R, C1772S, C1792K and C1827G. In some preferred embodiments, the engineered mouse TET2 enzyme shares at least 90%, 95%, 98%, 99% or 100% identity with SEQ ID NO: 16, with the proviso that the catalytic domain comprises one or more substitutions consisting of the following substitutions: A1286C, S1291C, K1332C, M1835C, E1837C, C1168Y, C1171P, C1185T, C1272R, C1772S, C1792K and C1827G.

In some particularly preferred embodiments, the engineered TET enzyme comprises a mouse TET3 catalytic domain comprising one or more substitutions, the one or more substitutions comprising, consisting essentially of, or consisting of one or more of the following substitutions: A1076C, I1112C, M1721C, E1726C, A975T, N1658S, and R1678K, wherein the positions are based on position number from full length mouse TET3 (SEQ ID NO: 10). In some preferred embodiments, the one or more substitutions of the mouse TET3 catalytic domain comprise, consist essentially of, or consist of 1, 2, 3, 4, 5, 6, or 7 of the substitutions in any combination. In some preferred embodiments, the catalytic domain of the engineered mouse TET3 enzyme has an N terminal portion that shares at least 90%, 95% or 98%, 99% or 100% sequence identity with SEQ ID NO:3 and a C terminal portion that shares at least 90%, 95%, 98%, 99%, or 100% sequence identity to SEQ ID NO:4, with the proviso that the catalytic domain comprises at least one substitution selected from the group consisting of A1076C, I1112C, M1721C, E1726C, A975T, N1658S, and R1678K and combinations thereof. In some preferred embodiments, the catalytic domain of the engineered mouse TET3 enzyme has an N terminal portion that shares at least 90%, 95% or 98%, 99% or 100% sequence identity with SEQ ID NO:3 and a C terminal portion that shares at least 90%, 95%, 98%, 99%, or 100% sequence identity to SEQ ID NO:4, with the proviso that the catalytic domain comprises one or more substitutions consisting of at least one substitution selected from the group consisting of A1076C, I1112C, M1721C, E1726C, A975T, N1658S, and R1678K and combinations thereof. In some preferred embodiments, the catalytic domain of the engineered mouse TET3 enzyme has an N terminal portion that has 100% sequence identity with SEQ ID NO:3 and a C terminal portion that has 100% sequence identity to SEQ ID NO: 4.

In some particularly preferred embodiments, the engineered TET enzyme comprises a Lygus hesperus TET2 catalytic domain one or more substitutions, the one or more substitutions comprising, consisting essentially of, or consisting of one or more of the following substitutions: A740C, K773C, Q1400C, V627P, A641T, N1337S, and H1357K, wherein the positions are based on position number from partial length Lygus hesperus TET2 (SEQ ID NO:11). In some preferred embodiments, the one or more substitutions of the Lygus hesperus TET2 catalytic domain comprise, consist essentially of, or consist of 1, 2, 3, 4, 5, 6 or 7 of the substitutions in any combination. In some preferred embodiments, the catalytic domain of the engineered Lygus hesperus TET2 enzyme has an N terminal portion that shares at least 90%, 95% or 98%, 99% or 100% sequence identity with SEQ ID NO:5 and a C terminal portion that shares at least 90%, 95%, 98%, 99%, or 100% sequence identity to SEQ ID NO: 6, with the proviso that the catalytic domain comprises at least one substitution selected from the group consisting of A740C, K773C, Q1400C, V627P, A641T, N1337S, and H1357K, and combinations thereof. In some preferred embodiments, the catalytic domain of the engineered Lygus hesperus TET2 enzyme has an N terminal portion that shares at least 90%, 95% or 98%, 99% or 100% sequence identity with SEQ ID NO:5 and a C terminal portion that shares at least 90%, 95%, 98%, 99%, or 100% sequence identity to SEQ ID NO: 6, with the proviso that the catalytic domain comprises one or more substitutions consisting of at least one substitution selected from the group consisting of A740C, K773C, Q1400C, V627P, A641T, N1337S, and H1357K, and combinations thereof. In some preferred embodiments, the catalytic domain of the engineered Lygus hesperus TET2 enzyme has an N terminal portion that has 100% sequence identity with SEQ ID NO:5 and a C terminal portion that has 100% sequence identity to SEQ ID NO:6.

In some particularly preferred embodiments, the engineered TET enzyme comprises a human TET2 catalytic domain comprising one or more substitutions, the one or more substitutions comprising, consisting essentially of, or consisting of one or more of the following substitutions: A1373C, K1409C, M1921C, E1923C, L1258P, A1272T, D1858S, and R1878K, wherein the positions are based on position number from full length human TET2 (SEQ ID NO: 12). In some preferred embodiments, the one or more substitutions of the human TET2 catalytic domain comprise, consist essentially of, or consist of 1, 2, 3, 4, 5, 6, 7, or 8 of the substitutions in any combination. In some preferred embodiments, the catalytic domain of the engineered human TET2 enzyme has an N terminal portion that shares at least 90%, 95% or 98%, 99% or 100% sequence identity with SEQ ID NO:7 and a C terminal portion that shares at least 90%, 95%, 98%, 99%, or 100% sequence identity to SEQ ID NO: 8, with the proviso that the catalytic domain comprises at least one substitution selected from the group consisting of A1373C, K1409C, M1921C, E1923C, L1258P, A1272T, D1858S, and R1878K and combinations thereof. In some preferred embodiments, the catalytic domain of the engineered human TET2 enzyme has an N terminal portion that shares at least 90%, 95% or 98%, 99% or 100% sequence identity with SEQ ID NO:7 and a C terminal portion that shares at least 90%, 95%, 98%, 99%, or 100% sequence identity to SEQ ID NO: 8, with the proviso that the catalytic domain comprises one or more substitutions consisting of at least one substitution selected from the group consisting of A1373C, K1409C, M1921C, E1923C, L1258P, A1272T, D1858S, and R1878K and combinations thereof. In some preferred embodiments, the catalytic domain of the engineered human TET2 enzyme has an N terminal portion that has 100% sequence identity with SEQ ID NO:7 and a C terminal portion that has 100% sequence identity to SEQ ID NO:8.

### 3. TET Orthologs

In other preferred embodiments, the present invention provides TET orthologs which have unexpected properties related to expression and/or activity as shown in the Examples. These TET orthologs may be selected from any TET enzyme from any species expressing a TET enzyme. For instance, in some embodiments, the TET orthologs may be selected from Aedes aegipty TET1, Anopheles gambia TET1, Drosophila ananassae TET1, Drosophila melanogaster TET1, Guinea pig TET1, Lama pacos TET1, Lygus hesperus TET2, Macaca fascicularis TET3, Mus pahari TET2, Rabbit TET2, Xenopus laevis TET1, Chanos chanos TET3, Sus scrofa TET3, Goldfish TET2 and Xenopus tropicalis TET3. In preferred embodiments, the TET orthologs are recombinant TET orthologs.

In some preferred embodiments, the orthologs may be engineered to include one or more modifications as described above. In some preferred embodiments, the orthologs are modified by truncation of the N terminus, most preferably up to the N terminal portion of the catalytic domain. In some preferred embodiments, the orthologs are modified to delete all or a portion of the LCR, for example at least 80%, 90%, 95%, 98% or 100% of the LCR in the particular ortholog. In some preferred embodiments, the LCR or portion thereof that has been deleted is replaced with a linker sequence. In some preferred embodiments, the linker sequence is a GS linker sequence. In some preferred embodiments, the orthologs are modified to include a C terminal truncation. In some preferred embodiments, the C terminal truncation may be from 10 to 77, 10 to 64, 10 to 54, 10 to 44, 10 to 34, or 10 to 24 amino acids in length. In some preferred embodiments, the orthologs may be modified to include one or more cysteine swaps. For example, in some embodiments, the substitutions may comprise, consist essentially of, or consist of 1) substitution of one or more non-cysteine amino acids in the catalytic domain of the TET ortholog with a cysteine; and/or 2) substitution of cysteine in the catalytic domain of the TET ortholog with a non-cysteine amino acid. In some preferred embodiments, substitution of the one or more non-cysteine amino acids in the catalytic domain of the TET ortholog with a cysteine is at a position that is occupied by a cysteine in the catalytic domain of a different TET isoform or ortholog. In some preferred embodiments, substitution of the one or more cysteines in the catalytic domain of the TET ortholog with a non-cysteine amino acid is at a position that is occupied by a non-cysteine amino acid in the catalytic domain of a different TET isoform or ortholog. In some preferred embodiments, the TET ortholog may be modified to include a His tag, Flag tag, or a combination thereof, preferably at the N terminus of the TET ortholog.

Accordingly, in some preferred embodiments, the present invention provides recombinant TET enzymes comprising a catalytic domain having at least 80%, 90%, 95%, 98%, 99% or 100% identity to the catalytic domain of a sequence selected from the group consisting of SEQ ID NO: 19 or 34 (Lygus hesperus TET2), SEQ ID NO:20 (Aedes aegipty TET1), SEQ ID NO:21 (Anopheles gambia TET1), SEQ ID NO:22 (Drosophila ananassae TET1), SEQ ID NO:23 (Drosophila melanogaster TET1), SEQ ID NO:24 (Guinea pig TET1), SEQ ID NO:25 (Lama pacos TET1), SEQ ID NO:26 (Macaca fascicularis TET3), SEQ ID NO:27 (Mus pahari TET2), SEQ ID NO:28 (Rabbit TET2), SEQ ID NO:29 (Xenopus laevis TET1), SEQ ID NO:30 (Chanos chanos TET3), SEQ ID NO:31 (Sus scrofa TET3), SEQ ID NO:32 (Goldfish TET2), and SEQ ID NO:33 (Xenopus tropicalis TET3). In some preferred embodiments, the TET ortholog catalyzes the oxidation of 5-methylcytosine (5mC) to 5-hydroxymethylcytosine (5hmC) and/or 5hmC to 5-formylcytosine (5fC) and/or 5-carboxycytosine (5caC). The sequences provided include the N terminal and C terminal portions of catalytic domains of the orthologs linked by a linker sequence.

It will be understood by a person of skill in the art that all or a portion of the LCR in these sequences may be identified and deleted and preferably replaced by a linker sequence. In some embodiments, the exemplified sequences include a GS linker in place of all or a portion of the LCR. It will be understood that when the LCR is specified as being deleted (all or a portion thereof), the percent identities defined above will be understood to refer to the N and C terminal portions of the catalytic domain that flank the LCR (or linker sequence when present). Thus, in some preferred embodiments, the present invention provides recombinant TET enzymes comprising a catalytic domain having at least 80%, 90%, 95%, 98%, 99% or 100% identity to the N and C terminal portions of a catalytic domain (e.g., the portion on either side of the linker sequence where present) of a sequence selected from the group consisting of SEQ ID NO: 19 or 34 (Lygus hesperus TET2), SEQ ID NO:20 (Aedes aegipty TET1), SEQ ID NO:21 (Anopheles gambia TET1), SEQ ID NO:22 (Drosophila ananassae TET1), SEQ ID NO:23 (Drosophila melanogaster TET1), SEQ ID NO:24 (Guinea pig TET1), SEQ ID NO:25 (Lama pacos TET1), SEQ ID NO:26 (Macaca fascicularis TET3), SEQ ID NO:27 (Mus pahari TET2), SEQ ID NO:28 (Rabbit TET2), SEQ ID NO: 29 (Xenopus laevis TET1), SEQ ID NO:30 (Chanos chanos TET3), SEQ ID NO:31 (Sus scrofa TET3), SEQ ID NO:32 (Goldfish TET2), and SEQ ID NO:33 (Xenopus tropicalis TET3). In some preferred embodiments, the TET ortholog catalyzes the oxidation of 5-methylcytosine (5mC) to 5-hydroxymethylcytosine (5hmC) and/or 5hmC to 5-formylcytosine (5fC) and/or 5-carboxycytosine (5caC).

### 4. Expression of Engineered TET Enzymes and Orthologs

In further embodiments, the present invention provides nucleic acids encoding the engineered TET enzymes and orthologs described above, vectors containing the nucleic acids, hosts cells containing the nucleic acids or vectors, and methods for the expression and production of the engineered TET enzymes and orthologs.

In some preferred embodiments, the nucleic acids encoding the engineered TET enzymes and orthologs may be employed for producing the engineered TET enzymes and orthologs by recombinant techniques. Thus, for example, the nucleic acids may be included in any one of a variety of expression vectors for expressing a polypeptide. In some embodiments of the present invention, vectors include, but are not limited to, retroviral vectors, chromosomal, nonchromosomal and synthetic DNA sequences (e.g., derivatives of SV40, bacterial plasmids, phage DNA, baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising a nucleic acid encoding an engineered TET enzyme or ortholog as described above. In some embodiments of the present invention, the constructs comprise a vector, such as a plasmid or viral vector, into which a sequence of the invention has been inserted, in a forward or reverse orientation. In still other embodiments, the nucleic acid sequence is assembled in appropriate phase with translation initiation and termination sequences. In preferred embodiments of the present invention, the appropriate nucleic acid (e.g., DNA) sequence is inserted into the vector using any of a variety of procedures. In general, the nucleic sequence is inserted into an appropriate restriction endonuclease site(s) by procedures known in the art. Appropriate cloning and expression vectors for use with prokaryotic and eukaryotic hosts are described by Sambrook, et al. (1989) Molecular Cloning: A Laboratory Manual, Second Edition, Cold Spring Harbor, N.Y.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the nucleic acid sequence in the expression vector is operatively linked to an appropriate expression control sequence(s) (promoter) to direct mRNA synthesis. Promoters useful in the present invention include, but are not limited to, viral long terminal repeats (LTR), the SV40 promoter, the E. coli lac and trp promoters, the phage lambda PL and PR promoters, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell.

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomyces cerevisiae, Schizosaccharomyces pombe, Drosophila S2 cells, Spodoptera Sf9 cells, Chinese hamster ovary (CHO) cells, COS-7 lines of monkey kidney fibroblasts, C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by retroviral transduction, calcium phosphate transfection, DEAE-Dextran mediated transfection, or electroporation (see, e.g., Davis et al. Basic Methods in Molecular Biology).

In some embodiments, the engineered TET polypeptides and orthologs are expressed in a baculovirus expression system (BVES). BVES systems are commercially available, for example from Expression Systems (Davis, CA) and ThermoFisher Scientific. In these embodiments, the nucleic acids are inserted into a baculovirus vector. First, the nucleic acid is cloned into a transfer plasmid, typically behind a promoter that can drive protein expression to high levels in insect cells. The nucleic acid that is to be expressed is typically flanked by AcMNPV DNA, e.g., the polyhedrin promoter on one side and a portion of the essential gene ORF1629 on the other. Insect cells are then co-transfected with a mixture of the transfer plasmid and parental AcMNPV DNA that has been linearized such that the parental polyhedrin gene and portion of ORF1629 are missing, rendering it non-infectious. The plasmid and parental DNA undergo homologous recombination to generate de novo recombinant baculoviruses. These baculoviruses are plated and individual plaques purified to isolate a single, pure plaque of recombinant baculovirus. This plaque is subsequently passaged through multiple rounds of insect cell infection to generate a high-titer stock and establish a working virus bank (WVB) that can be utilized for protein production.

Once a high-titer WVB has been established, it is used to infect insect cells and stimulate protein production. Cells are seeded in culture flasks (for small-scale production) or bioreactors (for large-scale production) and the WVB added to infect the insect cells when they are in their logarithmic growth phase. The baculoviruses reprogram the cellular machinery to produce the recombinant protein(s). Following protein expression (typically 48-96 hours post-infection), the cells and/or supernatant are harvested, depending on whether the product is intracellular or secreted, respectively.

In some embodiments of the present invention, following transformation of a suitable host strain and growth of the host strain to an appropriate cell density in media, protein is secreted and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

The present invention also provides methods for recovering and purifying the engineered TET enzymes and orthologs from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Thus, in some embodiments, the present invention provides improved methods for the expression and subsequent purification of active engineered TET enzymes and orthologs.

### 5. Use in Methylation Assays

5-Methylcytosine (5mC) and 5-hydroxymethylcytosine (5hmC) are the two major epigenetic marks found in the mammalian genome. 5hmC is generated from 5mC by TET enzymes. TET can further oxidize 5hmC to 5-formylcytosine (5fC) and 5-carboxylcytosine (5caC), which exists in much lower abundance in the mammalian genome compared to 5mC and 5hmC (10-fold to 100-fold lower than that of 5hmC). Together, 5mC and 5hmC play crucial roles in a broad range of biological processes from gene regulation to normal development. Aberrant DNA methylation and hydroxymethylation have been associated with various diseases and are well-accepted hallmarks of cancer. Therefore, the determination of 5mC and 5hmC in DNA sequence is not only important for basic research, but also is valuable for clinical applications, including diagnosis and therapy.

The ability of TET enzymes to oxidize methylated nucleotides is useful in assays designed to detect the presence of methylated nucleotides in a nucleic acid sample. Accordingly, in further embodiments, the present invention provides methods of assaying methylation of a target nucleic acid in a nucleic acid sample using the engineered TET enzymes and orthologs described above. The present invention is not limited to any particular methylation assay. The engineered TET enzymes and orthologs described above may be utilized in any assay where oxidation of methylated cytosines (e.g., 5mC or 5hmC) in a target nucleic acid is useful. For example, in some particularly preferred embodiments, the engineered TET enzymes and orthologs described above are used in Tet-Assisted Pyridine Borane Sequencing (TAPS). In other embodiments, the engineered TET enzymes and orthologs described above are used in Tet-Assisted Bisulfite Sequencing (TAB-Seq).

Embodiments of the present disclosure provide a bisulfite-free, base-resolution method for detecting 5-methylcytosine (5mC) and 5-hydroxymethylcytosine (5hmC) in a sequence (e.g., TAPS and associated methods TAPSB and CAPS, referred to collectively as TAPS), including for use with DNA obtained from blood samples (cellular DNA as well as cfDNA) and biopsies. As disclosed in International Pat. Publ. WO2019136413, U.S. Pat. Publ. 20200370114, U.S. Pat. Publ. 20210317519, International Pat. Publ. WO2021005537, International Pat. Publ. WO2022053872, International Pat. Publ. WO2021161192, and International Pat. Publ. WO2023007241 (each of which is incorporated herein by reference in its entirety), TAPS comprises the use of mild enzymatic and chemical reactions to detect 5mC and 5hmC directly and quantitatively at base-resolution without affecting unmodified cytosine. The present disclosure also provides methods to detect 5-formylcytosine (5fC) and 5-carboxylcytosine (5caC) at base resolution without affecting unmodified cytosine. Thus, the methods provided herein provide mapping of 5mC, 5hmC, 5fC and 5caC and overcome the disadvantages of previous methods such as bisulfite sequencing. In accordance with these embodiments, the methods of the present disclosure include the step of converting the 5mC and 5hmC (or just the 5mC if the 5hmC is blocked) to 5caC and/or 5fC. In some embodiments, this step comprises contacting the DNA or RNA sample with an engineered TET enzyme or ortholog as described above. The engineered TET enzyme catalyzes the transfer of an oxygen molecule to the C5 methyl group on 5mC resulting in the formation of 5-hydroxymethylcytosine (5hmC). The engineered TET enzyme further catalyzes the oxidation of 5hmC to 5fC and the oxidation of 5fC to form 5caC.

Methods of the present disclosure can also include the step of converting the 5caC and/or 5fC in a nucleic acid sample to DHU. In some embodiments, this step comprises contacting the DNA or RNA sample with a reducing agent including, for example, a borane reducing agent such as pyridine borane, 2-picoline borane (pic-BH3), borane, sodium borohydride, sodium cyanoborohydride, sodium triacetoxyborohydride, triethylamine borane or tri (t-butyl) amine borane.

In some embodiments, the methods of the present disclosure include identifying 5mC in a DNA sample (targeted DNA or whole-genome), and providing a quantitative measure for the frequency of the 5mC modification at each location where the modification was identified in the DNA. In some embodiments, the percentages of the T at each transition location provide a quantitative level of 5mC at each location in the DNA. In accordance with these embodiments, methods for identifying 5mC can include the use of a blocking group. In other embodiments, methods for identifying 5mC do not require the use of a blocking group.

When a blocking group is used to identify 5mC in a DNA without including 5hmC, the 5hmC in the sample is blocked so that it is not subject to conversion to 5caC and/or 5fC. In some embodiments, the 5hmC in the sample DNA are rendered non-reactive to the subsequent steps by adding a blocking group to the 5hmC. In one embodiment, the blocking group is a sugar, including a modified sugar, for example glucose or 6-azide-glucose (6-azido-6-deoxy-D-glucose). The sugar blocking group can be added to the hydroxymethyl group of 5hmC by contacting the DNA sample with uridine diphosphate (UDP)-sugar in the presence of one or more glucosyltransferase enzymes. In some embodiments, the glucosyltransferase is T4 bacteriophage β-glucosyltransferase (BGT), T4 bacteriophage α-glucosyltransferase (aGT), or derivatives and analogs thereof. βGT is an enzyme that catalyzes a chemical reaction in which a beta-D-glucosyl (glucose) residue is transferred from UDP-glucose to a 5-hydroxymethylcytosine residue in a nucleic acid.

In some embodiments, the methods of the present disclosure include identifying 5mC or 5hmC in a DNA sample (targeted DNA or whole-genome). In some embodiments, the method provides a quantitative measure for the frequency of the 5mC or 5hmC modifications at each location where the modifications were identified in the DNA. In some embodiments, the percentages of the T at each transition location provide a quantitative level of 5mC or 5hmC at each location in the DNA. In accordance with these embodiments, the method for identifying 5mC or 5hmC provides the location of 5mC and 5hmC, but does not distinguish between the two cytosine modifications. Rather, both 5mC and 5hmC are converted to DHU. The presence of DHU can be detected directly, or the modified DNA can be replicated, for instance by methods of the present disclosure, where the DHU is converted to T. In some embodiments, methods for identifying 5hmC include the use of a blocking group. In other embodiments, methods for identifying 5hmC do not require the use of a blocking group.

The present disclosure provides a method for identifying 5mC and identifying 5hmC in a DNA by performing the method for identifying 5mC on a first DNA sample, and performing the method for identifying 5mC or 5hmC on a second DNA sample. In some embodiments, the first and second DNA samples are derived from the same DNA sample. For example, the first and second samples may be separate aliquots taken from a sample comprising DNA to be analyzed (e.g., cellular DNA or cfDNA).

Because the 5mC and 5hmC (that is not blocked) are converted to 5fC and 5caC before conversion to DHU, any existing 5fC and 5caC in the DNA sample will be detected as 5mC and/or 5hmC. However, given the extremely low levels of 5fC and 5caC in genomic DNA under normal conditions, this will often be acceptable when analyzing methylation and hydroxymethylation in a DNA sample. The 5fC and 5caC signals can be eliminated by protecting the 5fC and 5caC from conversion to DHU by, for example, hydroxylamine conjugation and EDC coupling, respectively. In accordance with these embodiments, the method identifies the locations and percentages of 5hmC in the DNA through the comparison of 5mC locations and percentages with the locations and percentages of 5mC or 5hmC (together). Alternatively, the location and frequency of 5hmC modifications in a DNA can be measured directly.

In some embodiments, identifying 5fC and/or 5caC provides the location of 5fC and/or 5caC, but does not distinguish between these two cytosine modifications. Rather, both 5fC and 5caC are converted to DHU, which is detected by the methods described herein.

In some embodiments, the method includes identifying 5caC in a DNA sample (targeted DNA or whole-genome), and provides a quantitative measure for the frequency of the 5caC modification at each location where the modification was identified in the DNA. In some embodiments, the percentages of the T at each transition location provide a quantitative level of 5caC at each location in the DNA. In accordance with these embodiments, methods for identifying 5caC can include the use of a blocking group. In other embodiments, methods for identifying 5caC do not require the use of a blocking group.

In some embodiments, when the 5fC is blocked (and 5mC and 5hmC are not converted to DHU), the identification of 5caC in the DNA can occur. In some embodiments, adding a blocking group to the 5fC in the DNA sample comprises contacting the DNA with an aldehyde reactive compound including, for example, hydroxylamine derivatives, hydrazine derivatives, and hydrazide derivatives. Hydroxylamine derivatives include ashydroxylamine; hydroxylamine hydrochloride; hydroxylammonium acid sulfate; hydroxylamine phosphate; O-methylhydroxylamine; O-hexylhydroxylamine; O-pentylhydroxylamine; O-benzylhydroxylamine; and particularly, O-ethylhydroxylamine (EtONH2), O-alkylated or O-arylated hydroxylamine, acid or salts thereof. Hydrazine derivatives include N-alkylhydrazine, N-arylhydrazine, N-benzylhydrazine, N,N-dialkylhydrazine, N,N-diarylhydrazine, N,N-dibenzylhydrazine, N,N-alkylbenzylhydrazine, N,N-arylbenzylhydrazine, and N,N-alkylarylhydrazine. Hydrazide derivatives include-toluenesulfonylhydrazide, N-acylhydrazide, N,N-alkylacylhydrazide, N,N-benzylacylhydrazide, N,N-arylacylhydrazide, N-sulfonylhydrazide, N,N-alkylsulfonylhydrazide, N,N-benzylsulfonylhydrazide, and N,N-arylsulfonylhydrazide.

In some embodiments, the method includes identifying 5fC in a DNA sample (targeted DNA or whole-genome), and provides a quantitative measure for the frequency of the 5fC modification at each location where the modification was identified in the DNA. In some embodiments, the percentages of the T at each transition location provide a quantitative level of 5fC at each location in the DNA. In accordance with these embodiments, methods for identifying 5fC can include the use of a blocking group. In other embodiments, methods for identifying 5fC do not require the use of a blocking group.

In some embodiments, adding a blocking group to the 5caC in the DNA sample can be accomplished by (i) contacting the DNA sample with a coupling agent, for example a carboxylic acid derivatization reagent like carbodiimide derivatives such as 1-ethyl-3-(3-dimethylaminopropyl) carbodiimide (EDC) or N,N′-dicyclohexylcarbodiimide (DCC), and (ii) contacting the DNA sample with an amine, hydrazine or hydroxylamine compound. Thus, for example, 5caC can be blocked by treating the DNA sample with EDC and then benzylamine, ethylamine, or another amine to form an amide that blocks 5caC from conversion to DHU (e.g., by borane reduction).

In other embodiments, the engineered TET enzymes may be used in TAB-seq protocols. In TAB-Seq, 5hmC is preferably blocked with a sugar as described above for the TAPS protocol. 5mC in the blocked target nucleic acid is then oxidized to 5caC using an engineered TET enzyme or ortholog of the present invention. Following oxidation, the oxidized nucleic acid is treated with sodium bisulfite to reduce the 5caC to Uracil which is read as a T when sequenced. The TAB-Seq protocol may preferably combined with other sequencing protocols, for example, traditional bisulfite sequencing (methylC-Seq) to distinguish 5hmC from 5mC. In methylC-Seq, a target nucleic acid is treated with sodium bisulfite so that unmethylated cytosines are deaminated to uracil and read as a T whereas 5mC and 5hmC are read as C. Combination of methyl-Seq with TAB-Seq allows 5mC and 5hmC residues to be distinguished.

In still further embodiments, the engineered TET enzymes may be used in Enzymatic Methyl Sequencing (EM-seq) protocols. Enzymatic Methyl-seq (for New England Biolabs) is a two-step enzymatic conversion process to detect modified cytosines. The first step uses a TET enzyme, which can be an engineered TET enzyme or ortholog of the present invention, and an oxidation enhancer to protect modified cytosines from downstream deamination. The TET enzyme oxidizes 5mC and 5hmC through a cascade reaction into 5fC and 5caC as described above. In the EM-seq protocol, this step protects 5mC and 5hmC from deamination. As described above in relation to the other protocols, 5hmC can also be protected from deamination by glucosylation to form 5ghmC using the oxidation enhancer. The second enzymatic step uses APOBEC (Apolipoprotein B mRNA Editing Catalytic Polypeptide-like) enzyme to deaminate C to U but does not convert 5caC and 5ghmC.

In some preferred embodiments, the methods further comprise a sequencing step so that a methylation signature may be obtained. In some embodiments, the method includes isolating DNA (e.g., cellular or cfDNA) from a sample; preparing a sequencing library comprising the DNA; and performing TAPS or TAB-Seq on the sequencing library to obtain a methylation signature of the DNA. In some embodiments, the methylation signature is a whole-genome methylation signature.

In some embodiments, preparing the sequencing library comprises ligating sequencing adapters to the isolated DNA to facilitate performing a sequencing reaction. Suitable sequencing adapters for massively parallel sequencing technologies may be utilized. The present invention is not limited to any particular sequencing technology. In some preferred embodiments, sequencing technologies such as those provided by Illumina or Nanopore may be utilized. For example, suitable sequencing technologies for use in the present invention include, but are not limited to, those described in US Pat. Publ. 20100120098, US Pat. Publ. 20120208705, US Pat. Publ. 20120208724, International Pat. Publ. WO2012/061832, and US Pat. Publ. 2015/0368638, each of which is incorporated herein by reference in its entirety.

In some embodiments, the adapter comprises one or more sites that can hybridize to a primer. In some embodiments, an adapter comprises at least a first primer site. In some embodiments, an adapter comprises at least a first primer site and a second primer site. The orientation of the primer sites in such embodiments can be such that a primer hybridizing to the first primer site and a primer hybridizing to the second primer site are in the same orientation, or in different orientations. In one embodiment, the primer sequence in the linker can be complementary to a primer used for amplification. In another embodiment, the primer sequence is complementary to a primer used for sequencing.

In some embodiments, a linker can include a first primer site, a second primer site having a non-amplifiable site disposed therebetween. The non-amplifiable site is useful to block extension of a polynucleotide strand between the first and second primer sites, wherein the polynucleotide strand hybridizes to one of the primer sites. The non-amplifiable site can also be useful to prevent concatamers. Examples of non-amplifiable sites include a nucleotide analogue, non-nucleotide chemical moiety, amino-acid, peptide, and polypeptide. In some embodiments, a non-amplifiable site comprises a nucleotide analogue that does not significantly basepair with A, C, G or T.

Some embodiments include a linker comprising a first primer site, a second primer site having a fragmentation site disposed therebetween. Other embodiments can use a forked or Y-shaped adapter design useful for directional sequencing, as described in U.S. Pat. No. 7,741,463, which is incorporated herein by reference.

In some embodiments, the adapter may comprise an index or barcode sequence. In further preferred embodiments, the adapter may comprise a Unique Molecular Identifier (UMI).

In some embodiments, carrier nucleic acids or a mix of carrier nucleic acids (e.g., DNA) are added to the sequencing library prior to performing TAPS. Carrier nucleic acids can be any specific or non-specific DNA molecules (or nucleic acid derivatives thereof) that enhance one or more aspects of DNA recovery from a sample.

It is contemplated that DNA methylation signatures are useful for understanding basic biological processes and disease pathology as well as for disease detection. For example, methylation signatures/frequencies/markers etc. can be useful in understanding and studying gene regulation, genomic imprinting, differentiation, development, gene-environment interaction (e.g., smoking, nutrition), aging, numerous diseases and conditions (e.g., auto-immune diseases, cancer, cardiovascular diseases, CNS diseases, congenital diseases, infectious diseases, metabolic diseases and status, NIPT-related testing, etc.), for detecting and diagnosing cancer and other diseases and for monitoring transplants. In some embodiments, and as described herein, the method further comprises identifying at least one methylation biomarker from the DNA methylation signature (such as a whole-genome DNA methylation signature) and determining if the methylation biomarker differs from the methylation biomarker in a reference or control sequence. In some embodiments, the methylation biomarker comprises a differentially methylated region (DMR). In some embodiments, the method further comprises classifying the sample based on the DMR as compared to a reference DMR. In some embodiments, the reference DMR corresponds to a non-disease control, or a disease control.

In some embodiments, and as described herein, the method further comprises identifying at least one methylation biomarker from the DNA methylation signature, and determining a tissue-of-origin corresponding to the methylation biomarker. In some embodiments, the method further comprises classifying the sample based on the tissue-of-origin biomarker.

In some embodiments, and as described herein, the method further comprises identifying a DNA fragmentation profile, and determining whether the fragmentation profile is indicative of disease, such as cancer. In accordance with these embodiments, DNA fragmentation profile can be determined from TAPS sequencing data (e.g., read pair alignment positions).

In some embodiments, the method further comprises identifying at least one sequence variant in the DNA sample, and determining whether the sequence variant is indicative of disease, such as cancer. For example, in some embodiments, TAPS can also differentiate methylation from C-to-T genetic variants or single nucleotide polymorphisms (SNPs), and therefore, can be used to detect genetic variants. In some embodiments, methylations and C-to-T SNPs can result in different patterns in TAPS. For example, methylations can result in T/G reads in an original top strand/original bottom strand, and A/C reads in strands complementary to these. In some embodiments, C-to-T SNPs can result in T/A reads in an original top strand/original bottom strand and strands complementary to these. This further increases the utility of TAPS in providing both methylation information and genetic variants, and therefore mutations, in one experiment and sequencing run. This ability of the TAPS methods disclosed herein provides integration of genomic analysis with epigenetic analysis, and a substantial reduction of sequencing cost by eliminating the need to perform, for example, standard whole genome sequencing (WGS).

In accordance with the above embodiments, methods of the present disclosure include the use of TAPS or TAB-Seq to generate information pertaining to methylation signatures, methylation biomarkers, DNA fragment profiles, DNA sequence information (e.g., variants), and tissue-of-origin information in a single experiment to diagnose/detect a disease or other condition in a subject. As would be recognized by one of ordinary skill in the art based on the present disclosure, TAPS or TAB-Seq as disclosed herein can be used to generate any combination of methylation signatures, methylation biomarkers, DNA fragment profiles, DNA sequence information (e.g., variants), and tissue-of-origin information to diagnose/detect a disease or other condition in a subject. In some embodiments, a methylation signature can be obtained, and one or more of a methylation biomarker, a DNA fragment profile, DNA sequence information (e.g., variants), and tissue-of-origin information can also be obtained and used to diagnose/detect a disease or other condition in a subject. In some embodiments, the methylation status of a biomarker can be obtained, and one or more of a methylation signature, a DNA fragment profile, DNA sequence information (e.g., variants), and tissue-of-origin information can also be obtained and used to diagnose/detect a disease or other condition in a subject. In some embodiments, a DNA fragmentation profile can be obtained, and one or more of a methylation signature, a methylation biomarker, DNA sequence information (e.g., variants), and tissue-of-origin information can also be obtained and used to diagnose/detect a disease or other condition in a subject. In some embodiments, a DNA sequence variant can be identified, and one or more of a methylation signature, a methylation biomarker, a DNA fragment profile, and tissue-of-origin information can also be obtained and used to diagnose/detect a disease or other condition in a subject. In some embodiments, tissue-of-origin information can be obtained (e.g., from a whole genome DNA methylation signature), and one or more of the methylation signature, a methylation biomarker, a DNA fragment profile, and DNA sequence information (e.g., variants), can also be obtained and used to diagnose/detect a disease or other condition in a subject.

In some embodiments, performing TAPS or TAB-Seq on the sequencing library to obtain the whole-genome methylation signature comprises identifying 5mC modifications in the DNA and providing a quantitative measure for frequency of the 5mC modifications. In some embodiments, performing TAPS on the sequencing library to obtain the whole-genome methylation signature comprises identifying 5hmC modifications in the DNA and providing a quantitative measure for frequency of the 5hmC modifications. In some embodiments, performing TAPS on the sequencing library to obtain the whole-genome methylation signature comprises identifying 5caC modifications in the DNA and providing a quantitative measure for frequency of the 5caC modifications. In some embodiments, performing TAPS on the sequencing library to obtain the whole-genome methylation signature comprises identifying 5fC modifications in the DNA and providing a quantitative measure for frequency of the 5fC modifications.

As would be recognized by one of ordinary skill in the art based on the present disclosure, the methods described herein (e.g., TAPS, TAB-Seq and EM-Seq) can be used to diagnose/detect any type of cancer. Types of cancers that can be detected/diagnosed using the methods of the present disclosure include, but are not limited to, lung cancer, melanoma, colon cancer, colorectal cancer, neuroblastoma, breast cancer, prostate cancer, renal cell cancer, transitional cell carcinoma, cholangiocarcinoma, brain cancer, non-small cell lung cancer, pancreatic cancer, liver cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some embodiments, types of cancers or metastasizing forms of cancers that can be detected/diagnosed by the methods of the present disclosure include, but are not limited to, carcinoma, sarcoma, lymphoma, germ cell tumor and blastoma. In some embodiments, the cancer is invasive and/or metastatic cancer (e.g., stage II cancer, stage III cancer or stage IV cancer). In some embodiments, the cancer is an early-stage cancer (e.g., stage 0 cancer, stage I cancer), and/or is not invasive and/or metastatic cancer.

In accordance with these embodiments, the present disclosure provides methods for identifying the location of one or more of 5mC, 5hmC, 5caC and/or 5fC in a nucleic acid quantitatively with base-resolution without affecting the unmodified cytosine. In some embodiments, the nucleic acid is DNA. In some embodiments, the DNA is cfDNA (e.g., circulating cfDNA). In some embodiments, the nucleic acid is RNA. In some embodiments, a nucleic acid sample comprises a target nucleic acid that is DNA or a target nucleic acid that is RNA. In some embodiments, the methods are applied to a whole genome, and not limited to a specific target nucleic acid.

The nucleic acid may be any nucleic acid having cytosine modifications (i.e., 5mC, 5hmC, 5fC, and/or 5caC) but not limited to, DNA fragments and/or genomic DNA. The nucleic acid can be a single nucleic acid molecule in the sample, or may be the entire population of nucleic acid molecules in a sample, or any portion thereof (whole genome or a subset thereof). The nucleic acid can be the native nucleic acid from the source (e.g., cells, tissue samples, etc.) or can pre-converted into a high-throughput sequencing-ready form, for example by fragmentation, repair and ligation with adapters for sequencing. Thus, nucleic acids can comprise a plurality of nucleic acid sequences such that the methods described herein may be used to generate a library of target nucleic acid sequences that can be analyzed individually (e.g., by determining the sequence of individual targets) or in a group (e.g., by high-throughput or next generation sequencing methods).

The methods of the present disclosure can also include the step of amplifying the copy number of a modified nucleic acid by methods known in the art. When the modified nucleic acid is DNA, the copy number can be increased by, for example, PCR, cloning, and primer extension. The copy number of individual target DNAs can be amplified by PCR using primers specific for a particular target DNA sequence. Alternatively, a plurality of different modified target DNA sequences can be amplified by cloning into a DNA vector by standard techniques. In some embodiments, the copy number of a plurality of different modified target DNA sequences is increased by PCR to generate a library for next generation sequencing where, e.g., double-stranded adapter DNA has been previously ligated to the sample DNA (or to the modified sample DNA) and PCR is performed using primers complimentary to the adapter DNA.

In some embodiments, the method comprises the step of detecting the sequence of the modified nucleic acid. With respect to TAPS, the modified target DNA or RNA contains DHU at positions where one or more of 5mC, 5hmC, 5fC, and 5caC were present in the unmodified target DNA or RNA. DHU acts as a T in DNA replication and sequencing methods. Thus, the cytosine modifications can be detected by any direct or indirect method that identifies a C to T transition known in the art. Such methods include sequencing methods such as Sanger sequencing, microarray, and next generation sequencing methods. The C to T transition can also be detected by restriction enzyme analysis where the C to T transition abolishes or introduces a restriction endonuclease recognition sequence.

### 6. Kits or Systems

Embodiments of the present disclosure also provide systems or kits for oxidizing a methylated nucleotide (e.g., 5-methylcytosine (5mC) and 5-hydroxymethylcytosine (5hmC)). In some embodiments, the systems or kits comprise an engineered TET enzyme or ortholog as described in detail above.

The systems or kits may further comprise a borane reducing agent. In some embodiments, the borane reducing agent is selected from: pyridine borane, 2-picoline borane (pic-BH3), borane, sodium borohydride, sodium cyanoborohydride, sodium triacetoxyborohydride, diborane, decaborane, borane tetrahydrofuran, borane-dimethyl sulfide, borane-N,N-diisopropylethylamine, borane-2-chloropyridine, borane-aniline, N,N-dimethylamine borane, tert-butylamine borane sodium triacetoxyborohydride, boron hydride, hydrazine or dibutylamine borane, morpholine borane, borane-ammonia complex (BH3NH3), dicyclohexylamine borane, morpholine borane, 4-methylmorpholine borane, alkali and tetramethylamine boranes (e.g., NaBH4) and other-BH3 containing complexes and/or derivatives. In some embodiments, the reducing agent is pyridine borane and/or pic-BH3.

In some embodiments, the systems or kits further comprise a blocking group and/or a glucosyltransferase enzyme. In some embodiments, the blocking group is a sugar. In some embodiments, the sugar is a naturally-occurring sugar or a modified sugar, for example glucose or a modified glucose. In some embodiments, the blocking group functions with UDP linked to a sugar, for example UDP-glucose or UDP linked to a modified glucose in the presence of a glucosyltransferase enzyme, for example, T4 bacteriophage β-glucosyltransferase (BGT) and T4 bacteriophage α-glucosyltransferase (aGT) and derivatives and analogs thereof.

Such systems or kits may also be used for and comprise additional components necessary for the detection and identification of the methylated nucleotide. The systems or kits may further comprise sequencing reagents (e.g., primers, probes, nucleotides, buffers, control nucleic acid sequences, polymerases, etc.), restriction endonucleases, and the like for detecting the methylated nucleotide. The concepts, kits, and methods as described herein can be implemented on any system or instrument, including any manual, automated, or semi-automated system for sequencing reactions.

The systems or kits may include instructions for use in any of the methods described herein. Instructions included in the kit may be affixed to packaging material or may be included as a package insert. The instructions may be written or printed materials but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), etc. As used herein, the term “instructions” may include the address of an internet site that provides the instructions.

The kits provided herein are in suitable packaging. Suitable packaging includes, but is not limited to, vials, bottles, jars, flexible packaging, and the like. Kits optionally may provide additional components such as buffers and interpretive information. Normally, the kit comprises a container and a label or package insert(s) on or associated with the container. In some embodiments, the disclosure provides articles of manufacture comprising contents of the kits described above.

### EXAMPLES

The following are examples of the present invention and are not to be construed as limiting.

### Example 1

### 1.1 Comparison of Available TET Enzymes

This example describes the development of engineered TET enzymes amenable to high-volume, low-cost production that also exhibit the high performing functional characteristics essential for extremely sensitive assay applications such as TAPS. As TAPS is a new technology, others in the field have not faced the specific problem of identifying, engineering, or designing an enzyme optimized for TAPS.

The use of TET in the NGS arena has been limited to TAB-Seq (Yu et al. “Base-resolution analysis of 5-hydroxymethylcytosine in the mammalian genome” Cell. 2012 Jun. 8; 149 (6): 1368-80) and its incorporation in the NEB Next Enzymatic Methyl-seq Kit or EM-Seq. The TAB-seq method employs the Mouse TET1 catalytic domain comprising amino acids 1367-2039 as expressed from the gene GU079948 including the LCR. It was cloned with an N-terminal Flag-tag into pFastBac Dual vector (Invitrogen, cat: 10712024) and then expressed in the Bac-to-Bac baculovirus insect cell expression system. This is substantially the same as the preliminary Mouse TET1 construct described in more detail below. It was found to be unsuitable for commercial production by virtue of its propensity to aggregate and very poor expression levels in E. coli. This version was also generated in both mammalian and insect cells for analysis, however, these expression systems are more expensive to scale to production levels than E. coli, and also raise the complication of post translational modifications and resultant protein heterogeneity. Additionally, it has since been demonstrated that the engineered TET described herein has superior conversion rates (as demonstrated in the TAPS assay) and is very much more stable—as confirmed by thermofluor performed on Uncle (an all-in-one biologics stability screening platform from Unchained labs) and forced degradation studies, among other analyses as described in more detail below.

The NEB Next Enzymatic Methyl-seq Kit utilizes a Mouse TET2 enzyme and is advertised to provide a high-performance enzyme-based alternative to bisulfite conversion for methylome analysis using Illumina® sequencing. When the mouse TET2 enzyme from this kit was directly compared with the Mouse TET1 enzyme (as described above for the TAB-seq method), it was found that the Mouse TET1 performed better in TAPS and had higher conversion rates than the Mouse TET2 version from the NEB kit. Therefore, while the Mouse TET2 enzyme may be more amenable to commercial production, its limited performance relative to the Mouse TET1 enzyme would curtail the sensitivity advantages otherwise offered by the TAPS process.

Modifications to TET enzymes with a view to improving them functionally that have been reported, are limited to the expression of the Catalytic Domain (CD) in isolation (Tahiliani et al., “Conversion of 5-methylcytosine to 5-hydroxymethylcytosine in mammalian DNA by MLL partner TET1”. Science. 2009 May 15; 324 (5929): 930-5), removal or replacement of the LCR (Hu et al. “Crystal structure of TET2-DNA complex: insight into TET-mediated 5mc oxidation” Cell. 2013; 155:1545-1555), tagging of the protein (e.g., Raiber et al., “Mapping and elucidating the function of modified bases in DNA” Nat. Rev. Chem. 1, 0069 (2017)), and truncation of the C-terminus (Hu et al. 2013, supra; Hrit et al., (2018), “OGT binds a conserved C-terminal domain of TET1 to regulate TET1 activity and function in development”, eLife 7: e34870, Hu et al. “Structural insight into substrate preference for TET-mediated oxidation” Nature 527, 118-122 (2015)).

No solution currently exists that provides the yields and performance required for high volume commercial production of a sufficiently stringent, highly active and intrinsically stable TET enzyme, suitable for TAPs applications. A summary of results of testing constructs based on those previously described in the literature is provided in Table 2 below. The nearest equivalent to date, is the enzyme offered by NEB in their Next Enzymatic Methyl-seq Kit as discussed above. This commercially available Mouse TET2 isoform is described in patent US 2018/0171397 (incorporated herein by reference in its entirety). It achieves good soluble expression in E. coli having had its LCR removed to leave a “scar” sequence of flanking amino acids. However, the NEB enzyme is essentially the wild-type Mouse TET2 sequence and does not perform to the tolerances required of TAPs (as discussed above), either in an RUO device or in a clinical diagnostic setting—where high stability and processivity are required to achieve the highest levels of sensitivity (necessary for low titer clinical samples).

TABLE 2
Summary of testing TET enzymes with modifications described in the literature.

| | | | Expression | | | |
|---|---|---|---|---|---|---|
| Species | Isoform | Construct | System | Pros | Cons | Conclusion |
| Naegleria | | WT CD | Mammalian | No LCR | Processivity | Not suitable |
| gruberi | | | | | | |
| Mus | 1 | WT CD | E. coli | None | Poor yield / | Not suitable |
| Musculus | | | | | quality | |
| Mus | 1 | WT CD | Insect | Good TAPS | High COGs | Best |
| Musculus | | | | activity, OK | (Cost of | previously |
| | | | | yield | Goods) | available |
| | | | | | | version |
| Mus | 2 | WT CD | E. coli & | None | Poor yield / | Not suitable |
| Musculus | | | insect | | quality | |
| Mus | 3 | WT CD | E. coli & | None | Very poor | Not suitable |
| Musculus | | | insect | | yield / quality | |
| Homo | 1 | WT CD | E. coli & | None | Poor yield / | Not suitable |
| Sapiens | | | insect | | quality | |
| Homo | 2 | WT CD | E. coli & | None | Poor yield / | Not suitable |
| Sapiens | | | insect | | quality | |
| Homo | 3 | WT CD | E. coli & | None | Very poor | Not suitable |
| Sapiens | | | insect | | yield / quality | |
| Mus | 1 | WT CD - | Insect | Good TAPS | High COGs | Possible |
| Musculus | | LCR | | activity, OK | | alternative |
| | | | | yield | | to full- |
| | | | | | | length |
| | | | | | | version |
| Mus | 2 | WT CD - | E. coli | Good E. coli | Lower TAPS | MmTET1 |
| Musculus | | LCR | | yield & low | activity than | better in |
| | | | | COGs | MmTET1 | TAPS |
| Mus | 3 | WT CD - | E. coli & | Good TAPS | Poor yield | Unsuitable, |
| Musculus | | LCR | insect | activity | | needs |
| | | | | | | engineering |
| Homo | 1 | WT CD - | Insect | OK activity | Poor yields | MmTET1 |
| Sapiens | | LCR | | | | better |
| | | | | | | option |
| Homo | 2 | WT CD - | Insect | OK activity | Poor yields | MmTET1 |
| Sapiens | | LCR | | | | better |
| | | | | | | option |
| Homo | 3 | WT CD - | E. coli & | Good TAPS | Poor yield | Unsuitable, |
| Sapiens | | LCR | insect | activity | | needs |
| | | | | | | engineering |

Yield is expressed as mg of pure active protein per ml of cell culture harvested.
Poor yield: <1 mg/ml
OK yield: >1 mg/ml but <10 mg/ml
Good yield: >10 mg/ml

1.2 Mouse TET1 is Poorly Expressed in E. coli.

For expression and purification of mouse TET1 (also referred to as MmTET1 or mTET) the enzyme was modified with a dual N-terminal tag of Hexa-Histidine (His-tag) followed by a Flag-tag. The His-tag was to act as the primary purification option via Immobilized Metal Affinity Chromatography (IMAC), while the Flag-tag gave the option of anti-Flag-tag ImmunoPrecipitation (IP) or “pull-down”. The dual tags also provided two means to track the protein via, for example, Western blotting (i.e., anti-His or anti-Flag primary antibodies) and a means to remove the tag if desired, as the Flag-tag sequence was also an Enterokinase recognition site. The His-Flag tagged MmTET1 was cloned into an expression vector and expressed in E. coli.

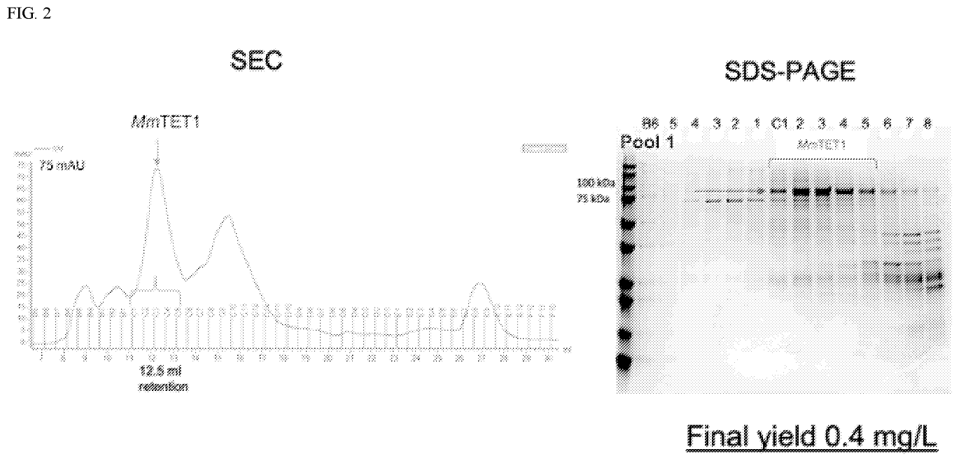

The His-Flag tagged soluble TET was captured via Ni IMAC then applied to SEC (Size Exclusion Chromatography). The SEC profile, provided in FIG. 2, illustrates both the low expression level (the main peak only reaches 75 mAU) and the relative impurity of the prep following IMAC. This impurity is in part a factor of the relative enrichment of non-specifically binding host proteins when the target protein levels are so low, but it is also due to aggregates and proteolysis products of the TET co-purifying with the intact enzyme. A typical final yield for the MmTET1 Full Length (FL) CD expressed and extracted from E. coli was <1 mg/L of total cell culture, making economic production at large-scale unviable. See FIG. 2.

1.3 Deletion of LCR is not Sufficient to Improve Soluble Expression in E. coli

Active TET Catalytic Domains from various species have been heterogeneously expressed while retaining their central LCRs (see e.g., Tahiliani et al., supra). However, constructs retaining their LCRs have generally expressed with low abundance (Dickson and Golding, “Low Complexity Regions in Mammalian Proteins are Associated with Low Protein Abundance and High Transcript Abundance” Molecular Biology and Evolution (39): 5 (2022)), and there is a direct, yet not fully understood, relation to disorder, aggregation and flexibility-characteristics which can limit soluble heterologous expression, and also prevent crystallization (Mier P, et al. “Disentangling the complexity of low complexity proteins”. Brief Bioinform. 2020 Mar. 23; 21 (2): 458-472).

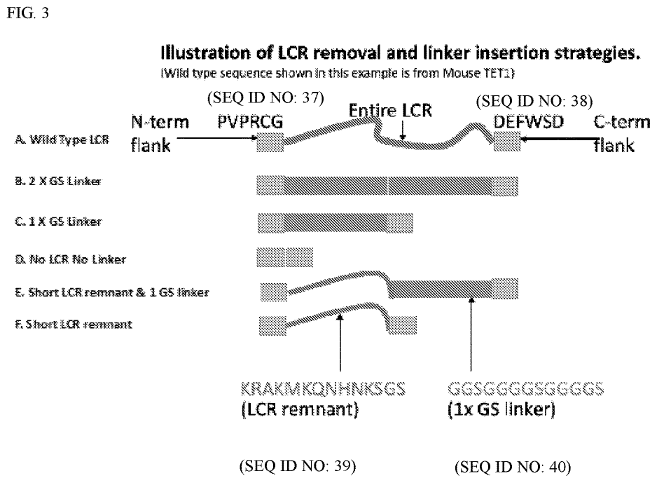

Typically, when engineering proteins or domains for high soluble yields and/or crystallization, any unstructured regions may be removed or replaced with linkers. Other groups have already shown that removal or replacement of the LCR is beneficial in terms of TET CD crystallization (Hue et al. 2013, supra), consequently this was the first approach applied for the current Example. Whole and partial deletion of the LCR was tested along with insertion of single and double GS linkers-flexible linkers with sequences consisting primarily of stretches of Gly and Ser residues (Chen et al., “Fusion protein linkers: property, design and functionality”. Adv Drug Deliv Rev. 2013 October; 65 (10): 1357-69). The options tested are shown in FIG. 3. After testing these constructs, option C in FIG. 3 was selected, which replaced the entire LCR with a single GS linker. Additionally, several creative alternatives to the GS linker replacement of the LCR, comprising a variety of sequences predicted (by in silico modelling) to form helices, were tested. These various modifications generally had negative effects of their own or made no substantial improvement to the enzyme's performance. In summary, even with the LCR removed or replaced, MmTET1 soluble expression was poor in E. coli.

### 1.4 C Terminal Truncation of MmTET2 Catalytic Domain (CD)

It was next observed that the Mammalian TET2 isoform, especially that derived from mouse, did express with high soluble yields in E. coli, especially once the LCR was replaced by a GS linker. However, further engineering and optimization was required to obtain high performance in TAPS, reduce degradation propensity and improve stability. The latter would ultimately limit manufacturing methods, product formulation, storage duration and conditions, transportation modes (e.g., blue ice/dry ice), functional performance and ultimate application (e.g., it may be unsuitable for highly stringent healthcare applications or for use with robotic platforms).

Additionally, the extreme C-terminal region of the Catalytic Domain is reported to have specific functions in vivo (Hrit et al., supra) related to the associated O-GlcNAc transferase (OGT) protein—at least in the Mammalian TET1 isoform and probably in all three isoforms (Deplus et al. (2013) “TET2 and TET3 regulate GlcNAcylation and H3K4 methylation through OGT and SET1/COMPASS” EMBO J. 2013 Mar. 6; 32 (5): 645-55; Chen et al. “TET2 promotes histone O-GlcNAcylation during gene transcription” Nature. 2013; 493:561-564; Ito et al. “TET3-OGT interaction increases the stability and the presence of OGT in chromatin” Genes to Cells. (2014) 19:52-65). However, the lack of intrinsic structure in this region makes it vulnerable to proteolytic degradation, which was observed when expressed in E. coli. Consequently, there are benefits in terms of protein stability, preparative homogeneity and structural utility (e.g., crystallographic analyses) to constructs that are truncated in this region (Hu et al. “Structural insight into substrate preference for TET-mediated oxidation” Nature 527, 118-122 (2015)).

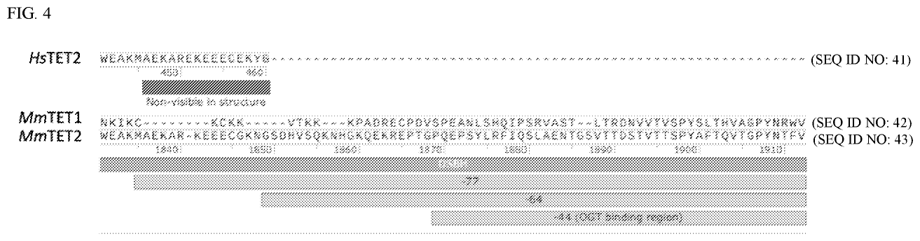

Informed by this, C-terminal truncations of an MmTET2 CD construct were made, as highlighted in FIG. 4, showing the MmTET2 and MmTET1 sequences aligned to a published structure (PDB 5D9Y-see Table 3) of HsTET2 CD deltaLCR. Removing the final 77 C-terminal residues generated a truncation corresponding to the final electron density of this structure, while removing the last 64 residues mimics the precise C-terminus of the construct used to obtain the structure. Thus, both C-terminal truncations aimed to eliminate this apparently unstructured/disordered region of the protein.

TABLE 3
3D structures of TET Catalytic Domains in the Protein
Data Bank (PDB; on the world wide web at rcsb.org)

| PDB Ref | Species | Isoform | Ligand |
|---|---|---|---|
| 4NM6 | Hs | TET2 | DNA Oligo |
| 5D9Y | Hs | TET2 | 5fC oligo |
| 5DEU | Hs | TET2 | 5hmC oligo |
| 7NE3 | Hs | TET2 | DNA Oligo |
| 7NE6 | Hs | TET2 | “Unfavorable” |
| | | | (GpC) DNA Oligo |
| 4LT5 | Ng | TET1 | 5mC oligo |
| 5CG8 | Ng | TET1 | 5mC oligo |
| 5CG9 | Ng | TET1 | 5mC oligo |
| 7CY4 | Cr | — | Apoenzyme |
| 7CY5 | Cr | — | Ascorbate (Vitamin C) |
| 7CY6 | Cr | — | 5mC oligo |

Hs = Homo sapiens
Ng = Naegleria gruberi
Cr = Chlamydomonas reinhardtii

It was observed that truncation of the C-terminus beyond removal of the final 64 residues may become increasingly detrimental as removal of the 77th residue is approached, since beyond this, the structural integrity of the final helix is impinged upon.

The C-terminus was also truncated at the final 44 residues (highlighted in FIG. 4) corresponding to removal of the OGT binding region of the protein (as described in Hrit et al, supra). Truncation at this position had the desired positive impact on integrity and stability while also generating the most active of the various C-terminal truncation options as shown in Table 4. Table 4 provides data on the different MmTET2 enzymes, including yield, activity in a TAPS assay, and stability. It is possible that truncations removing still fewer residues (i.e., fewer than 44) may have diminishing benefits, as more of the proteolytically labile region would be retained. However, the general benefits were clearly demonstrated, in terms of an active and stable TET enzyme suitable for manufacture, of truncations within the range of the final 77 residues, and the data in Table 4 below, illustrates the relative impact of the key truncation positions on activity and stability.

TABLE 4
Comparison of MmTET2 constructs of
varying C-terminal configurations.

| | | Activity | | |
|---|---|---|---|---|
| | Yield | TAPS % 1 h | Stability | Tagg/ |
| Description | mg/L | (16 μg Mm TET2) | Tm/° C. | ° C. |
| Mm TET2 deltaLCR | 30 | 83.6 | 67 | 55.3 |
| Mm TET2 deltaLCR C-44 | 30 | 90 | 64.2 | 45.9 |
| Mm TET2 deltaLCR C-64 | 30 | 83.5 | 64.1 | 54.6 |
| Mm TET2 deltaLCR C-77 | 20 | 41 | 63.8 | 54.6 |

C-44″ = C-terminal deletion of last 44 residues. Similarly, C-64 and C-77 refer to deletions of 64 & 77 residues respectively.

Specifically, Table 4 provides results for four MmTET2 CD constructs, each having a His-Flag N-terminal tag and their LCR replaced by a GS linker. They differed at the C-terminus which was variously either Full Length (as wild-type) or truncated by the noted number of residues (i.e., −44, −64 and −77). The most active construct was observed to be the MmTET2 deltaLCR C-44. There appeared to be a minor sacrifice of thermal stability relative to the wild-type but this was rewarded by the elimination of proteolytic degradation. Note that truncation by 77 residues halved activity, and further truncation is known to eliminate activity (Hu et al. 2013, supra).

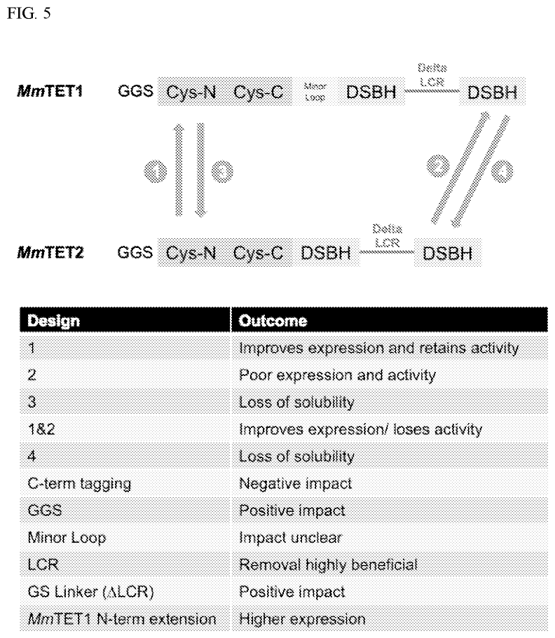

The described modifications of MmTET2 eliminated the degradation previously observed and improved activity. However, despite this, the situation remained that the most active isoform (TET1) had poor expression characteristics (even with the LCR removed or C-terminus truncated) while the best expressing isoform (TET2) had a less useful activity profile than isoform 1 even once modified. Numerous approaches were attempted to either improve TET1 handling characteristics or to improve TET2 performance characteristics. Rational modifications were considered, including domain swaps, and mutations at the surface, active site, metal binding and DNA binding regions. These are summarized in FIG. 5. These efforts provided some interesting insights into the factors most impacting soluble expression and activity, but none resolved key issues.

### 1.5 Engineering of TET by Cysteine Swaps Improves Manufacturing Characteristics and Activity in TAPS

In order to address the problems noted with prior engineered TET enzymes described above, a different engineering approach was sought. It was observed that non-conserved cysteines influence isoformal character. Thus, type-2 TET (i.e., MmTET2) was modified such that its Cys distribution mimicked type-1 TET (i.e., MmTET1). As shown below, it was observed that type-1 TET enzymatic performance was achieved in type-2 TET with these modifications.

A preferred engineered MmTET2 was thus developed and is represented by the following sequence:

MmTET2_N-His-Flag_CD_deltaLCR_1xGS_CysSwap_-44 C-term (also
referred to herein as TET v1.0; SEQ ID NO: 16)
MGHHHHHHDYKDDDDKGGSQSQNGKCEGCNPDKDEAPYYTHLGAGPDVAAIRTLMEERYGEK
GKAIRIEKVIYTGKEGKSSQGCPIAKWVYRRSSEEEKLLCLVRVRPNHTCETAVMVIAIMLW
DGIPKLLASELYSELTDILGKYGIPTNRRCSQNETRNCTCQGENPETCGASFSFGCSWSMYY
NGCKFARSKKPRKFRLHGAEPKEEERLGSHLONLATVIAPIYKKLAPDAYNNQVEFEHQAPD
CRLGLKEGRPFSGVTCCLDFCAHSHRDQQNMPNGSTVVVTLNREDNREVGACPEDEQFHVLP
MYIIAPEDEFGSTEGQEKKIRMGSIEVLQSFRRRRVIRIGGGGGSGGGGSGGGGSVQEIEYW
SDSEHNFQDPSIGGVAIAPTHGSILIECAKKEVHATTKVNDPDRNHPTRISLVLYRHKNLFL
PKHGLALWEAKCACKARKEEECGKNGSDHVSQKNHGKQEKREPTG

This engineered MmTET2 comprises the following features:
(1) Mutations: C1168Y, C1171P, C1185T, C1272R, A1286C, S1291C, K1322C, C1772S, C1792K, C1827G, M1835C, E1837C
(2) Addition of N-terminal 6×His and Flag tags (HHHHHHDYKDDDDK (SEQ ID NO: 35)
(3) Addition of a GGS immediately after the flag tag and preceding Q1042
(4) Deletion of the LCR between E1373-A1754 inclusive
(5) Addition of a GS linker GGGGSGGGGSGGGGS (SEQ ID NO: 13) replacing E1373-A1754 inclusive
(6) Truncation of the native Mouse TET2 C-terminus such that it ends at G1868
(7) Truncations of the C-terminus from its extreme end (position) up to and including (position—e 77 residues from the end) are viable constructs with preferred embodiments being C-termini ending at: (position delta 44) or (position delta 65).

Table 5 below shows the positions in the catalytic domain of mouse TET2 at which cysteine residues were replaced with non-cysteine residues or non-cysteine residues were replaced cysteine residues to replicate the cysteine distribution in the catalytic domain of mouse TET1. This engineered MmTET2 was tested as described below. Table 5 also provides positions at which cysteine swaps may be made in MmTET3 (see, e.g., SEQ ID NO: 17), HsTET2 (see, e.g., SEQ ID NO: 18), and Lygus hesperus TET2 (see, e.g., SEQ ID NO: 19). Column 2 “Cysteine Number” refers to the position in the CDs of either MmTET1 or MmTET2 isoforms where a Cysteine is present in one or the other of the isoforms. Column 3 provides the absolute numbering of the positions of interest, the numbers being taken from the sp-Q4JK59 Uniprot deposition (SEQ ID NO: 9) for the entire MmTET2 protein (i.e., residues 1-1912 inclusive). Column 4 lists the mutations made in the preferred embodiment using numbering from the sp-Q4JK59 Uniprot deposition. Column 5 lists all the positions of interest and the mutations made using numbering from SEQ ID NO: 16. Column 6 lists cysteine swap substitutions using numbering based on SEQ ID NO:10 (full-length MmTET3). Column 7 lists cysteine swap substitutions for using numbering based on SEQ ID NO:11 (partial length Lygus hesperus TET2). Column 8 lists cysteine swap substitutions for using numbering based SEQ ID NO:12 (full-length HsTET2). It will be noted that except with respect to Column 5, the numbering of the substitutions is based on the position number in the listed sequence. The actual position in the engineered enzyme may differ due to the inclusion of features such as purification tags, a truncated, N terminus, a truncated C terminus, a deleted LCR and substitution of a linker sequence for the LCR.

TABLE 5
Cysteine swaps in selected engineered TET enzymes

| | | | | Equivalent residue | | | |
|---|---|---|---|---|---|---|---|
| | | sp-Q4JK59 MmTET2 | Cys Swaps | numbers in | | | |
| | | wt FL CD | made in | CD of preferred | | Cys Swaps | |
| | | Numbering as for | Preferred | embodiment (i.e. | | made in Lygus | Cys Swaps |
| Mu- | | entire TET gene, | Embodiment | numbering for the His- | Cys Swaps made in | hesperus TET2 | made in hTET2 |
| tation | Cysteine | i.e. starts upstream | (whole gene | Flag_MmTET2 CD with LCR | mTET3 (whole gene | (partial gene | (whole gene |
| Number | Number | of the CXXC Region | numbering) | replaced by GS linker) | numbering) | numbering) | numbering) |
| | C_1 | 1048 | | 26 | | | |
| | C_2 | 1051 | | 29 | | | |
| | C_3 | 1106 | | 84 | | | |
| | C_4 | 1124 | | 102 | | | |
| | C_5 | 1134 | | 112 | | | |
| 1 | C_6 | 1168 | C1168Y | C146Y | Y958Y (no change) | Y624Y (no | Y1255Y (no |
| | | | | | | change) | change) |
| 2 | C_7 | 1171 | C1171P | C149P | P961P (no change) | V627P | L1258P |
| | C_8 | 1176 | | 154 | | | |
| | C_9 | 1184 | | 162 | | | |
| 3 | C_10 | 1185 | C1185T | C163T | A975T | A641T | A1272T |
| | C_11 | 1186 | | 164 | | | |
| | C_12 | 1194 | | 172 | | | |
| | C_13 | 1202 | | 180 | | | |
| | C_14 | 1211 | | 189 | | | |
| | C_15 | 1271 | | 249 | | | |
| 4 | C_16 | 1272 | C1272R | C250R | R1062R (no change) | R726R (no | R1359R (no |
| | | | | | | change) | change) |
| 5 | C_17 | 1286 | A1286C | A264C | A1076C | A740C | A1373C |
| | C_18 | 1287 | | 265 | | | |
| 6 | C_19 | 1291 | S1291C | S269C | C1081C (no change) | C745C (no | C1378C (no |
| | | | | | | change) | change) |
| 7 | C_20 | 1322 | K1322C | K300C | I1112C | K773C | K1409C |
| | C_21 | 1378 | | I349-GS-linker-V366 | | | |
| | C_22 | LCR_1402 | LCR replaced by | GS_Linker | LCR replaced by | LCR replaced by | LCR replaced by |
| | C_23 | LCR_1489 | linker | VIRIGGGGGSGGGGSGGGGSVQEI | linker | linker | linker |
| | | | | (SEQ ID NO: 36) | | | |
| 8 | C_24 | 1772 | C1772S | C383S | N1658S | N1337S | D1858S |
| | C_25 | 1789 | | 400 | | | |
| 9 | C_26 | 1792 | C1792K | C403K | R1678K | H1357K | R1878K |
| 10 | C_27 | 1827 | C1827G | C438G | G1713G (no change) | G1392G (no | G1913G (no |
| | | | | | | change) | change) |
| 11 | C_28 | 1835 | M1835C | M446C | M1721C | Q1400C | M1921C |
| 12 | C_29 | 1837 | E1837C | E448C | E1726C | C1402C (no | E1923C |
| | | | | | | change) | |
| | C_30 | 1845 | | 456 | | | |

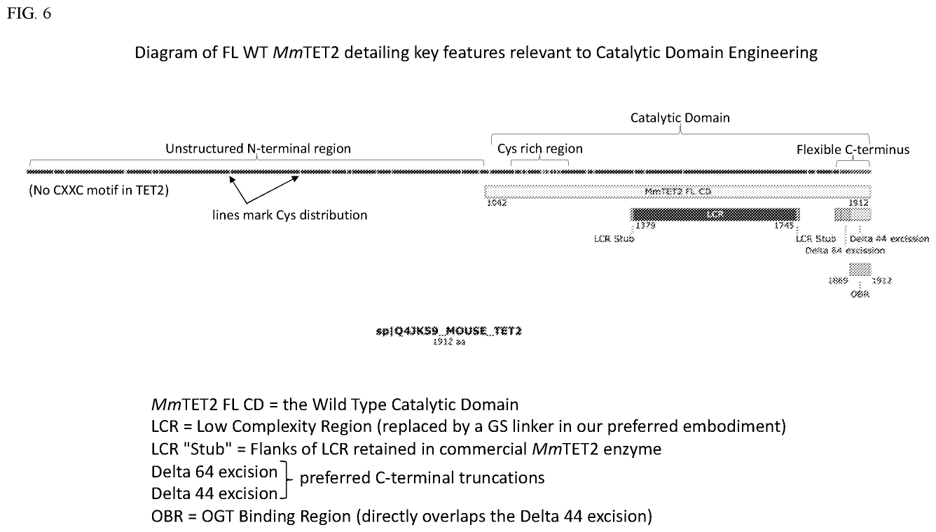

FIG. 6 provides a detailed scheme of MmTET2 features relating to Catalytic Domain (CD) engineering (see, e.g., SEQ ID NO: 16). TET enzymes contain a Catalytic Domain (CD) comprising a conserved double-stranded β-helix (DSBH) domain, a cysteine-rich domain, and binding sites for the cofactors Fe (II) and 2-oxoglutarate (2-OG) that together form the core catalytic region in the C-terminus. With respect to SEQ ID NO: 16, the Mouse TET2 CD sequence spans the equivalent of Q1042 to G1868 of Uniprot: Q4JK59. The Cys residues in the CD of both the type 1 TET and type 2 TET isoforms of the Mouse enzyme were mapped, noting that the conserved Cys residues each had identifiable functions (e.g., relating to Zinc Finger formation or Iron Binding). The non-conserved residues were then explored and swapped relative to each other across the isoforms, such that where there was a Cys in isoform 1 the equivalent isoform 2 position was replaced with a Cys, and where there was a Cys in the native isoform 2 it was replaced with the equivalent non-Cys residue from isoform 1. In this way, an isoform 2 protein was derived with the Cys distribution pattern of isoform 1. For MmTET2, 12 such “Cys-swaps” were made, as detailed in Table 5 above. Table 5 also provides predicted Cys swaps for MmTET3, HsTET2, and Lygus hesperus TET2.

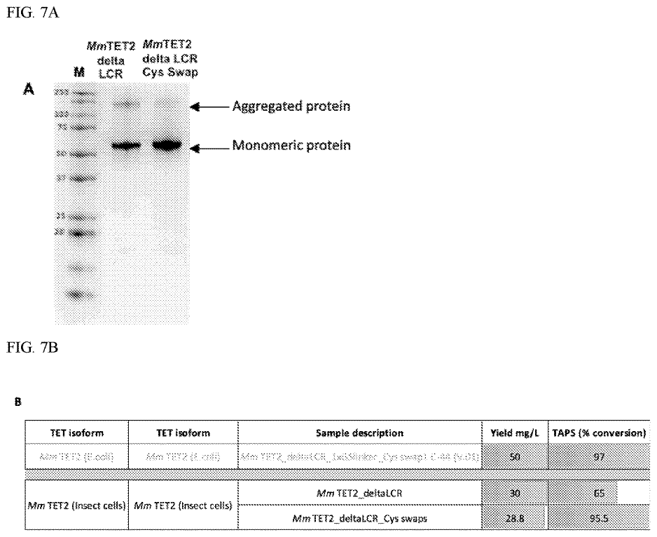

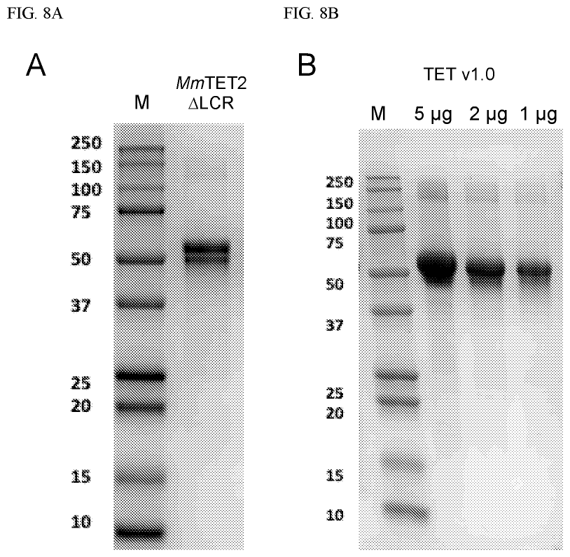

The cysteine swaps, as applied to the engineered MmTET2 construct, improved their activity and developability (i.e., improved solubility and stability, while reducing aggregation propensity) over the unmutated versions without compromising yield in either insect or bacterial expression systems. FIG. 7 provides data showing that the cysteine swaps as applied to MmTET2 (i.e., SEQ ID NO: 16, also referred to herein as TET v1.0) expressed in insect cells improved yield and enzyme activity in TAPS. FIG. 8 provides SDS-PAGE analysis of MmTET2 with a deleted LCR as compared to TET v1.0, both produced in E. coli. The TET v1.0 demonstrated a more homogenous preparation from E. coli as assayed by SDS-PAGE, which showed a doublet for MmTET2 with a deleted LCR and a single resolved band for TET v1.0.

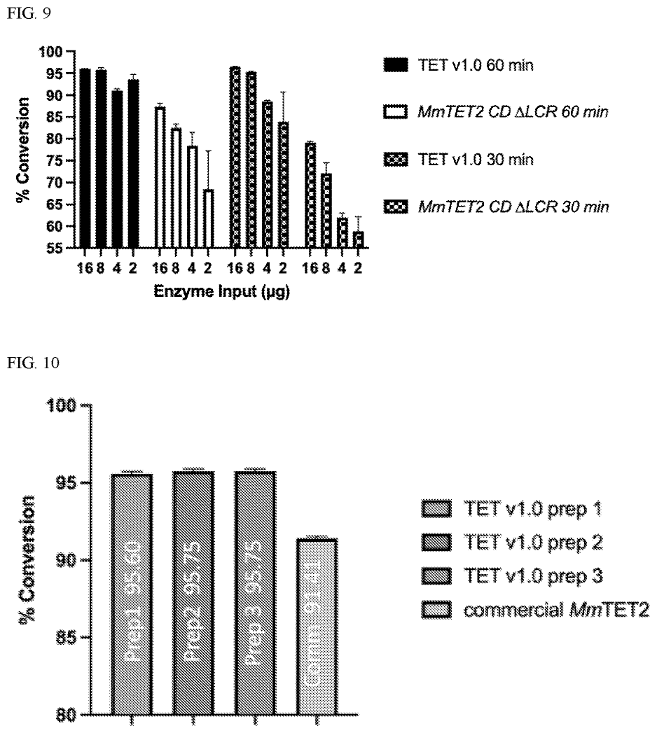

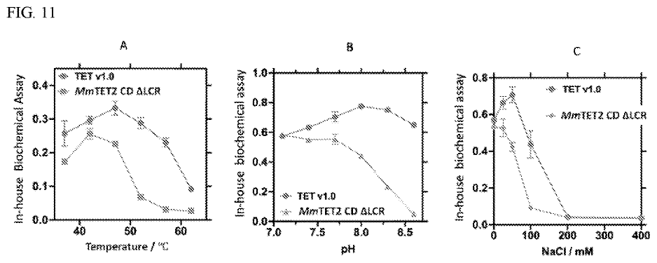

TET v1.0 was also assayed for activity in TAPS as compared to various other TET enzymes. First, TET v1.0 was compared to wild-type MmTET2 CD with a deleted LCR (MmTET2 CD ALCR). The difference between the two enzymes is that the TET v1.0 contains cysteine swaps while the MmTET2 CD ALCR does not contain cysteine swaps. The oxidation step was conducted for either 30 or 60 minutes followed by borane reduction and sequencing. The data, which is provided in FIG. 9, shows that TET v1.0 demonstrated increased % conversion in the TAPS assay as compared to MmTET2 CD ALCR. The % conversion shown in FIG. 9 is the % of methylated Cytosines detected in a fully methylated Lambda sample. Next, three preparations of TET v1.0 were compared to a commercial MmTET2 (New England Biolabs) for activity in TAPS as measured by % conversion. The data is presented in FIG. 10, which shows that TET v1.0 had an increased % conversion (above 95%) as compared to the commercial enzyme (about 91%). Next, a TET enzyme activity assay was used to compare the activity of TET v1.0 to MmTET2 CD ALCR over a range pf temperature, pH, and NaCl concentrations. The data is presented in FIG. 11 and shows that the cysteine swap enhances the biochemical activity of the enzyme over wider temperatures (FIG. 11A), a range of pH conditions (FIG. 11B) and a range of NaCl concentrations (FIG. 11C), when compared to the wild-type protein. Table 6 below provides a summary of conversion rates, activity and stability of TET v1.0 as compared to MmTET1 CD, MmTET2 ALCR with or without C terminal truncations, and a MmTET2 ALCR with cysteine swaps but without a C terminal truncation. As can be seen, TET v1.0 demonstrated the best combination of yield, activity and stability.

TABLE. 6
Table of conversion rates, activity & stability of TET enzymes.

| | | | | Enzymatic | Enzymatic Activity_ |
|---|---|---|---|---|---|
| | | | Puri- | Activity | Degradation Study |
| | | | fication | Oxidation | Oxidation of methyl-C |
| | | | Yield | of methyl-C | meaured by TAPS (%) |
| | | Expression | Yield | meaured by | 1 day 37 °C/fresh |
| Construct | Protein Features | system | (mg/L) | TAPS (%) | sample |
| Mm TET1 CD FL | Full length catalytic | Insect | 10 | 91.4 ± 1.09 | 0.79 |
| | Domain, including the | Cells | | (n = 10) | |
| | LCR | | | | |
| commercial | ΔLCR | E. coli | N.A. | 92.4 ± 0.4 | 0.99 |
| Mm TET2 | | | | (n = 18) | |
| deltaLCR | | | | | |
| In-house Mm | ΔLCR = GGGGS₃ | E. coli | 50 | 91.8 ± 0.6 | 0.69 |
| TET2deltaLCR_1 | | | | (n = 24) | |
| In-house Mm | ΔLCR = GGGGS₃, | E. coli | 50 | 92.7 ± 0.5 | 0.77 |
| TET2deltaLCR_2 | C-terminus | | | (n = 10) | |
| | truncation −64 | | | | |
| In-house Mm | ΔLCR = GGGGS₃, | E. coli | 50 | 82.2 ± 1.3 | 0.89 |
| TET2deltaLCR_3 | C-terminus | | | (n = 4) | |
| | truncation −44 | | | | |
| In-house Mm | ΔLCR = GGGGS₃, Non- | E. coli | 50 | 93.4 ± 0.4 | 0.92 |
| TET2deltaLCR_4 | conserved Cys residues | | | (n = 4) | |
| | swap | | | | |
| In-house | ΔLCR = GGGGS₃, Non- | E. coli | 50 | 94.1 ± 0.3 | 0.95 |
| Mm | conserved Cys | | | (n = 33) | |
| TET2deltaLCR_ | residues swap, C- | | | | |
| V1.0 | terminus truncation −44 | | | | |

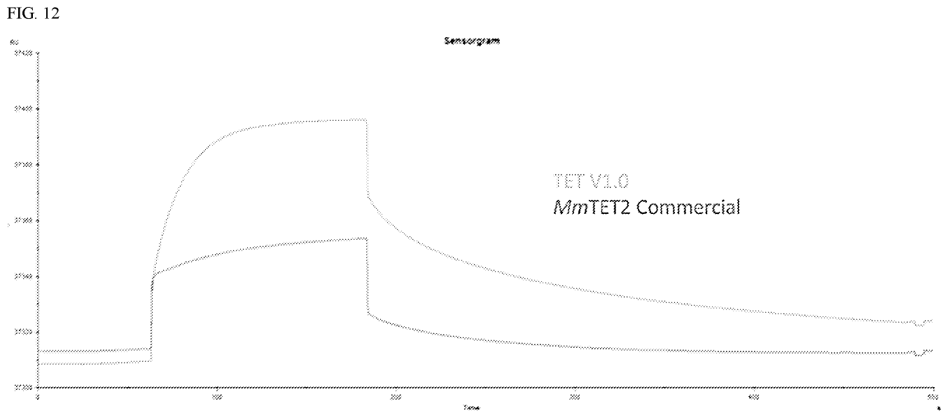

The DNA binding affinity of TET v1.0 as compared to commercial MmTET2 was also examined. The data is provided in FIG. 12. The SPR sensorgram shows a direct comparison of TET v1.0 in light grey (higher signal sensorgram), with the commercially available MmTET2 in black (lower signal sensorgram). DNA was immobilized on the SPR chip surface and equimolar solutions of the enzymes were flowed across it. The degree of enzyme binding to DNA was measured in Response Units (RU) plotted on the Y axis. TET v1.0 has greater DNA binding affinity than the commercial enzyme. Changes in the region involved in the DNA recognition of Human TET2 are reported to almost abolish the biochemical activity of the enzyme (Hu et al. 2013, supra). These data show an increased ability of TET v1.0 to bind DNA compared to the wild-type MmTET2. This different DNA recognition could be one of the determining factors in conferring an increased biochemical activity. One hypothesis is that the Cys swap may confer the ability to retain DNA substrate for longer, thereby increasing target base residence time in the active site, ensuring completion of the catalytic cycle.

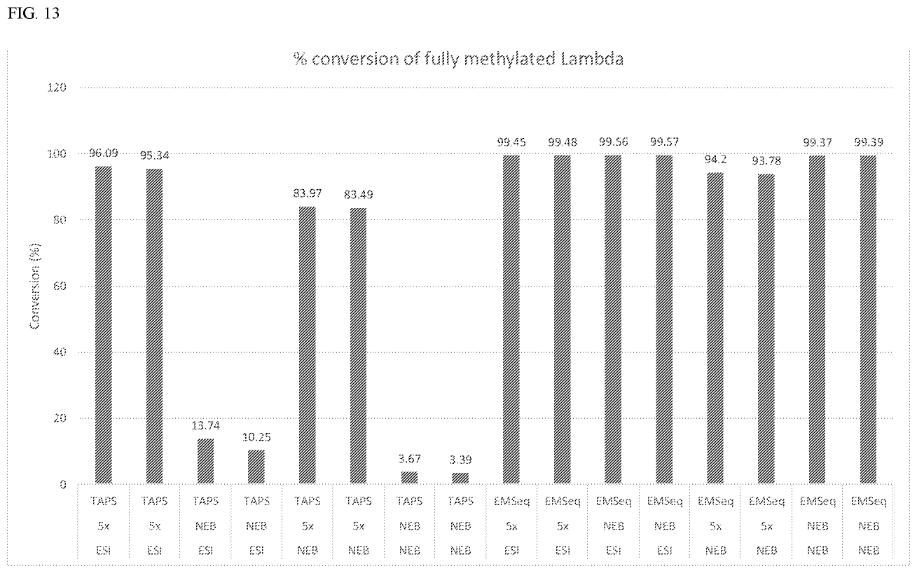

Finally, TET v1.0 was tested alongside the New England BioLabs (NEB) mTET2 in both the TAPS assay and the Enzymatic Methyl-seq (Em-seq) assay from NEB. The results are shown in FIG. 13. As demonstrated in FIG. 13, TET v1.0 significantly outperforms the NEB mTET2 in the TAPS assay when used with either 5× buffer (Exact Sciences buffer) or NEB buffer. TET v1.0 also performs equivalent or better in the Em-Seq assay when used in Exact Sciences buffer or the NEB buffer.

The cysteine swap strategy described can be applied to any TET CD to improve activity and developability characteristics, in specific embodiments they may be applied to MmTET2, MmTET3, Lygus hesperus TET2, as well as other orthologs. The data show that the distribution of non-conserved cysteines may influence distinct isoformal characteristics (e.g., improved performance in NGS assays such as TAPS) by means unknown-possibly via interactions with substrate DNA.

Taken together, these data show that TET enzymes modified by cysteine swaps exhibited improved production yield, stability and activity as compared to previously engineered TET and commercial TET polymerases.

### Example 2

Seventy-four orthologs of TET1, TET2 and TET3 from higher and lower species were screened. The constructs comprised the CD of the TET orthologs including the LCR and did not have a C terminal truncation. Based upon initial screening in a 1 ml HTP format, the following TET enzymes were selected for further testing based on their expression levels: SEQ ID NO:19 (Lygus hesperus TET2), SEQ ID NO:20 (Aedes aegipty TET1), SEQ ID NO: 21 (Anopheles gambia TET1), SEQ ID NO:22 (Drosophila ananassae TET1), SEQ ID NO: 23 (Drosophila melanogaster TET1), SEQ ID NO:24 (Guinea pig TET1), SEQ ID NO: 25 (Lama pacos TET1), SEQ ID NO:26 (Macaca fascicularis TET3), SEQ ID NO:27 (Mus pahari TET2), SEQ ID NO:28 (Rabbit TET2), SEQ ID NO:29 (Xenopus laevis TET1), SEQ ID NO:30 (Chanos chanos TET3), SEQ ID NO:31 (Sus scrofa TET3), SEQ ID NO:32 (Goldfish TET2) and SEQ ID NO:33 (Xenopus tropicalis TET3). Data on these constructs, which had either significant expression or activity via an in-house assay, are reported in Table 7.

TABLE 7
Activity of Orthologs of TET1, TET2 and TET3 Enzymes

| | | Activity | Activity | Activity | | | | | |
|---|---|---|---|---|---|---|---|---|---|
| | | 100 ml scale | 100 ml scale | 1 ml scale | | | | | |
| | | 3 μg protein | 6 μg protein | 5 μl input | | | | | |
| Isoform | Species | 45 min. | 45 min. | 60 min. | | | Coomassie band intensity | | |
| TET1 | Aedes | 0.044441 | 0.122697 | | | | | | |
| | aegipty | | | | | | | | |
| TET1 | Anopheles | 0.040992 | 0.07155 | | | | | | |
| | gambia | | | | | | | | |
| TET1 | Drosophila | 0.040274 | 0.089228 | | | | | | |
| | ananassae | | | | | | | | |
| TET1 | Drosophila | 0.041742 | 0.094034 | | | | | | |
| | melanogaster | | | | | | | | |
| TET1 | Guinea pig | 0.047222 | 0.064957 | | | | | | |
| TET1 | Lama | 0.064568 | 0.067278 | | | | | | |
| | pacos | | | | | | | | |
| TET2 | Lygus | 0.765592 | 0.6399 | 0.448 | 0.676 | 0.583 | High | High | High |
| | hesperus | | | | | | | | |
| TET3 | Macaca | 0.183004 | 0.104028 | | | | | | |
| | fascicularis | | | | | | | | |
| TET2 | Mus pahari | 0.061517 | 0.067301 | 0.08 | 0.255 | 0.081 | High | High | Medium |
| TET2 | Rabbit | 0.550457 | 0.146332 | | | | | | |
| TET1 | Xenopus | | | 0.18 | 0.218 | 0.179 | Low | Low | Low |
| | laevis | | | | | | | | |
| TET3 | Chanos | | | 0.075 | | 0.075 | Low | Low | Low |
| | chanos | | | | | | | | |
| TET3 | Sus scrofa | | | | 0.182 | | Low | Low | Low |
| TET2 | Goldfish | | | 0.189 | | | Medium | Medium | Medium |
| TET3 | Xenopus | | | 0.111 | | | Low | Low | Low |
| | tropicalis | | | | | | | | |

Lygus hesperus TET2 had especially promising results and has low sequence identity (about 40%) to MmTET2. It should be noted that all orthologs tested contained a full length C-terminal region and it is expected that truncation of the C terminal region as described for MmTET2 will additionally improve expression, stability and activity. Additionally, no cysteine swaps were incorporated in the orthologs tested in the example. Cysteine swaps are also expected to improve expression, stability and activity.

### Example 3

Expression of different constructs using a baculovirus expression system was examined. The data is presented in Table 8. The sequences encoding the TET constructs were cloned into a baculovirus expression vector and transfected into insect cells for expression. Unexpectedly, a MmTET3 ALCR construct, which produced insoluble protein in E. coli, showed high solubility when expressed in insect cells. Good expression for MmTET1 and MmTET2 ALCR constructs in this system as well as better performance of the enzymes in general was also observed. In particular, TET v1.0 produced in this system had a 97% conversion rate in a TAPS assay. Finally, a MmTET2 CD construct containing the LCR showed unexpectedly high activity in TAPS despite low expression and purity. This data demonstrates that good yields of highly active engineered TET enzymes can be obtained in the baculovirus expression system.

TABLE 8
Yield (mg/L) and activity (via an in-house assay) and TAPS % methylation
in a baculovirus expression system for MmTET1, MmTET2, and MmTET3
constructs with and without LCR and cysteine swaps

| TET | | Yield | Activity | TAPS (% | |
|---|---|---|---|---|---|
| isoform | Sample description | mg/L | assay | methylation) | Comment |
| TET2 | MmTET2_ΔLCR_1xGSlinker_Cys | 50 | active | 97 | V.01 |
| (E.coli) | swap1 (from E. coli) | | | | |
| TET1 | MmTET1_ML+_CD_FL | 8 | active | 94 | active only in |
| | | | | | R2 buffer |
| | MmTET1_ML+_ΔLCR_1xGSlinker | 3 | active | 92 | active only in |
| | | | | | R2 buffer |
| | MmTET1_ML+_CD_FL | 4.5 | active | 89 | active only in |
| | | | | | R2 buffer |
| TET2 | MmTET2_ΔLCR | 11 | active | 33 | active in R2 |
| | | | | | and 5X |
| | MmTET2_ΔLCR | 8 | active | 94.5 | active in R2 |
| | | | | | and 5X |
| | MmTET2_ΔLCR_1xGSlinker | 30 | active | 65 | active in R2 |
| | | | | | and 5X |
| | MmTET2_ΔLCR_1xGSlinker_Cys | 28.8 | active | 95.5 | active in R2 |
| | swap1 | | | | and 5X |
| | MmTET2_CD_FL | 1 | active | 96.5 | active in R2 |
| | | | | | and 5X |
| TET3 | MmTET3_ΔLCR_longer N term | 4 | not active | | |
| | MmTET3_ΔLCR | 9 | active | 95.2 | active in R2 |
| | | | | | and 5X |

| | | |
|---|---|---|
| MmTET3_ΔLCR longer N term | 6 | not active |
| chimera with mTET2 | | |
| MmTET3_ΔLCR chimera with | 20 | not active |
| MmTET2 | | |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. An engineered Ten-Eleven Translocase (TET) enzyme comprising a TET catalytic domain comprising at least one substitution mutation selected from the group consisting of:
substitution of a non-cysteine amino acid in the catalytic domain with a cysteine;
substitution of cysteine in the catalytic domain with a non-cysteine amino acid; and
combinations thereof;
wherein the engineered TET enzyme catalyzes the oxidation of 5-methylcytosine (5mC) to 5-hydroxymethylcytosine (5hmC) and/or 5hmC to 5-formylcytosine (5fC) and/or 5-carboxycytosine (5caC).

2. The engineered TET enzyme of claim 1, wherein the at least one non-cysteine amino acid that is substituted with a cysteine occurs at a position in the catalytic domain of the TET enzyme that is occupied by a cysteine in an isoform or ortholog of the TET enzyme.

3. The engineered TET enzyme of claim 2, wherein the TET catalytic domain is a mouse TET2 catalytic domain and the isoform is mouse TET1.

4. The engineered TET enzyme of claim 3, wherein the at least one non-cysteine amino acid that is substituted with a cysteine is at a position selected from the group consisting of 1286, 1291, 1322, 1835, and 1837 and combinations thereof, wherein the position numbers are based on position numbers for full length mouse TET2.

5. The engineered TET enzyme of claim 4, wherein the substitutions are selected from the group consisting of A1286C, S1291C, K1332C, M1835C and E1837C substitutions and combinations thereof, wherein the position numbers are based on position numbers for full length mouse TET2.

6. The engineered TET enzyme of any one of claims 1 to 5, wherein the at least one cysteine in the catalytic domain that is substituted with a non-cysteine amino acid occurs at a position in the catalytic domain of the TET enzyme that is occupied by a non-cysteine in an isoform of the TET enzyme.

7. The engineered TET enzyme of claim 6, wherein the TET catalytic domain is a mouse TET2 catalytic domain and the isoform is a mouse TET1 enzyme.

8. The engineered TET enzyme of claim 7, wherein the at least one cysteine that is substituted with a non-cysteine amino acid is at a position selected from the group consisting of 1168, 1171, 1185, 1272, 1772, 1792 and 1827 and combinations thereof, wherein the position numbers are based on position numbers for full length mouse TET2.

9. The engineered TET enzyme of claim 8, wherein the substitutions are selected from the group consisting of C1168Y, C1171P, C1185T, C1272R, C1772S, C1792K and C1827G substitutions and combinations thereof, wherein the position numbers are based on position numbers for full length mouse TET2.

10. The engineered TET enzyme of any one of claims 1 to 9, wherein the catalytic domain of the engineered TET enzyme has an N terminal portion that shares at least 80% sequence identity with SEQ ID NO: 1 and a C terminal portion that shares at least 80% sequence identity to SEQ ID NO:2, with the proviso that the catalytic domain comprises at least one substitution selected from the group consisting of A1286C, S1291C, K1332C, M1835C, E1837C, C1168Y, C1171P, C1185T, C1272R, C1772S, C1792K and C1827G substitutions and combinations thereof, wherein the position numbers are based on position numbers for full length mouse TET2.

11. The engineered TET enzyme of claim 10, wherein the N terminal portion of the catalytic domain corresponds to SEQ ID NO: 1 and the C terminal portion of the catalytic domain corresponds to SEQ ID NO:2.

12. The engineered TET enzyme of any one of claims 10 to 11, where the N terminal portion of the catalytic domain and the C terminal portion of the catalytic domain are connected by a heterologous linker sequence.

13. The engineered TET enzyme of claim 2, wherein the TET catalytic domain is a mouse TET3 catalytic domain and the isoform is mouse TET1.

14. The engineered TET enzyme of claim 13, wherein the at least one non-cysteine amino acid that is substituted with a cysteine is at a position selected from the group consisting of 1076, 1112, 1721, and 1726 and combinations thereof, wherein the position numbers are based on position numbers for full length mouse TET3.

15. The engineered TET enzyme of claim 14, wherein the substitutions are selected from the group consisting of A1076C, I1112C, M1721C, and E1726C substitutions and combinations thereof, wherein the position numbers are based on position numbers for full length mouse TET3.

16. The engineered TET enzyme of any one of claims 13 to 15, further comprising an amino acid substitution at a position selected from the group consisting of 975, 1658, and 1678 and combinations thereof, wherein the position numbers are based on position numbers for full length mouse TET3.

17. The engineered TET enzyme of claim 16, wherein the substitutions are selected from the group consisting of A975T, N1658S, and R1678K substitutions and combinations thereof, wherein the position numbers are based on position numbers for full length mouse TET3.

18. The engineered TET enzyme of any one of claims 13 to 17, wherein the catalytic domain of the engineered TET enzyme has an N terminal portion that shares at least 80% sequence identity with SEQ ID NO:3 and a C terminal portion that shares at least 80% sequence identity to SEQ ID NO:4, with the proviso that the catalytic domain comprises at least 1 substitution selected from the group consisting of A1076C, I1112C, M1721C, E1726C, A975T, N1658S, and R1678K substitutions and combinations thereof, wherein the position numbers are based on position numbers for full length mouse TET3.

19. The engineered TET enzyme of claim 18, wherein the N terminal portion of the catalytic domain corresponds to SEQ ID NO:3 and the C terminal portion of the catalytic domain corresponds to SEQ ID NO:4.

20. The engineered TET enzyme of any one of claims 18 to 19, where the N terminal portion of the catalytic domain and the C terminal portion of the catalytic domain are connected by a heterologous linker sequence.

21. The engineered TET enzyme of claim 2, wherein the TET catalytic domain is a Lygus hesperus TET2 catalytic domain and the ortholog is mouse TET1.

22. The engineered TET enzyme of claim 21, wherein the at least one non-cysteine amino acid that is substituted with a cysteine is at a position selected from the group consisting of 740, 773, and 1400 and combinations thereof, wherein the position numbers are based on position numbers for partial Lygus hesperus TET2.

23. The engineered TET enzyme of claim 22, wherein the substitutions are selected from the group consisting of A740C, K773C, and Q1400C substitutions and combinations thereof, wherein the position numbers are based on position numbers for partial length Lygus hesperus TET2.

24. The engineered TET enzyme of any one of claims 21 to 23, further comprising an amino acid substitution at a position selected from the group consisting of 627, 641, 1337, and 1357 and combinations thereof, wherein the position numbers are based on position numbers for partial length Lygus hesperus TET2.

25. The engineered TET enzyme of claim 24, wherein the substitutions are selected from the group consisting of V627P, A641T, N1337S, and H1357K substitutions and combinations thereof, wherein the position numbers are based on position numbers for full length Lygus hesperus TET2.

26. The engineered TET enzyme of any one of claims 21 to 25, wherein the catalytic domain of the engineered TET enzyme has an N terminal portion that shares at least 80% sequence identity with SEQ ID NO:5 and a C terminal portion that shares at least 80% sequence identity to SEQ ID NO:6, with the proviso that the catalytic domain comprises at least 1 substitution selected from the group consisting of A740C, K773C, Q1400C, V627P, A641T, N1337S, and H1357K, substitutions and combinations thereof, wherein the position numbers are based on position numbers for partial length Lygus hesperus TET2.

27. The engineered TET enzyme of claim 26, wherein the N terminal portion of the catalytic domain corresponds to SEQ ID NO:5 and the C terminal portion of the catalytic domain corresponds to SEQ ID NO:6.

28. The engineered TET enzyme of any one of claims 26 to 27, where the N terminal portion of the catalytic domain and the C terminal portion of the catalytic domain are connected by a heterologous linker sequence.

29. The engineered TET enzyme of claim 2, wherein the TET catalytic domain is a human TET2 catalytic domain and the ortholog is mouse TET1.

30. The engineered TET enzyme of claim 29, wherein the at least one non-cysteine amino acid that is substituted with a cysteine is at a position selected from the group consisting of 1373, 1409, 1921, and 1923 and combinations thereof, wherein the position numbers are based on position numbers for full length human TET2.

31. The engineered TET enzyme of claim 30, wherein the substitutions are selected from the group consisting of A1373C, K1409C, M1921C, and E1923C substitutions and combinations thereof, wherein the position numbers are based on position numbers for full length human TET2.

32. The engineered TET enzyme of any one of claims 29 to 31, further comprising an amino acid substitution at a position selected from the group consisting of 1258, 1272, 1858, and 1878 and combinations thereof, wherein the position numbers are based on position numbers for full length human TET2.

33. The engineered TET enzyme of claim 32, wherein the substitutions are selected from the group consisting of L1258P, A1272T, D1858S, and R1878K substitutions and combinations thereof, wherein the position numbers are based on position numbers for full length human TET2.

34. The engineered TET enzyme of any one of claims 29 to 33, wherein the catalytic domain of the engineered TET enzyme has an N terminal portion that shares at least 80% sequence identity with SEQ ID NO:7 and a C terminal portion that shares at least 80% sequence identity to SEQ ID NO: 8 with the proviso that the catalytic domain comprises at least 1 substitution selected from the group consisting of A1373C, K1409C, M1921C, E1923C, L1258P, A1272T, D1858S, and R1878K substitutions and combinations thereof, wherein the position numbers are based on position numbers for full length human TET2 and the engineered TET enzyme catalyzes the oxidation of 5-methylcytosine.

35. The engineered TET enzyme of claim 34, wherein the N terminal portion of the catalytic domain corresponds to SEQ ID NO: 7 and the C terminal portion of the catalytic domain corresponds to SEQ ID NO:8.

36. The engineered TET enzyme of any one of claims 34 to 35, where the N terminal portion of the catalytic domain and the C terminal portion of the catalytic domain are connected by a heterologous linker sequence.

37. The engineered TET enzyme of any one of claims 1 to 36, wherein the TET enzyme has an N terminal truncation, optionally wherein the N-terminal truncation extends to the N-terminus of the catalytic domain.

38. The engineered TET enzyme of anyone of claims 1 to 37, wherein the TET enzyme has a C terminal truncation.

39. The engineered TET enzyme of claim 38, wherein the C terminal truncation is up to and including 77 amino acids.

40. The engineered TET enzyme of claim 38, wherein the C terminal truncation is up to and including 64 amino acids.

41. The engineered TET enzyme of claim 38, wherein the C terminal truncation is up to and including 44 amino acids.

42. The engineered TET enzyme of any one of claims 1 to 41, wherein all or a portion of the Low Complexity Region (LCR) is deleted, optionally wherein the LCR is substituted with a heterologous linker sequence.

43. The engineered TET enzyme of any one of claims 1 to 42, wherein the enzyme comprises at least one purification tag selected from the group consisting of a his tag and a FLAG tag and combinations thereof.

44. A nucleic acid encoding the engineered TET enzyme of any one of claims 1 to 43.

45. The nucleic acid of claim 44, further comprising a promoter in operable association with the nucleic acid encoding the engineered TET enzyme.

46. A vector comprising the nucleic acid of any one of claims 44 to 45.

47. A host cell comprising the nucleic acid or vector of any one of claims 44 to 46.

48. The host cell of claim 47, wherein the host cell is a prokaryotic host cell.

49. The host cell of claim 47, wherein the host cell is a eukaryotic host cell.

50. A method of producing an engineered TET enzyme comprising:
culturing the host cell of any one of claims 47 to 49 under conditions such that the engineered TET enzyme is expressed; and
isolating the expressed engineered TET enzyme.

51. A method of producing an engineered TET enzyme comprising:
introducing a baculovirus vector comprising a nucleic acid sequence according to any one of claims 44 to 45 into an insect cell free expression system under conditions such that the engineered TET enzyme is expressed; and
isolating the expressed engineered TET enzyme.

52. A method of producing a recombinant TET enzyme comprising:
introducing a baculovirus vector comprising a nucleic acid sequence encoding a TET enzyme into insect cell expression system under conditions such that the recombinant TET enzyme is expressed; and
isolating the expressed recombinant TET enzyme.

53. The method of claim 52, wherein the recombinant TET enzyme is a recombinant mTET1, mTET2, or mTET3 enzyme or an engineered TET enzyme or ortholog as described in any of claims 1 to 43.

54. The method of any one of claims 52 to 53, wherein the recombinant TET enzyme does not comprise an LCR.

55. The method any one of claims 52 to 54, wherein the LCR is substituted with a heterologous linker sequence.

56. The method of any one of claims 52 to 55, wherein the recombinant TET enzyme has an N-terminal truncation.

57. The method of claim 56, wherein the N-terminal truncation extends to the N terminus of the catalytic domain of the recombinant TET enzyme.

58. A recombinant TET enzyme produced by the method of any one of claims 52 to 57.

59. A recombinant TET enzyme comprising a catalytic domain having at least 80%, 90%, 95%, 98% or 100% identity to the catalytic domain of a sequence selected from the group consisting of SEQ ID NOs: 19 to 33 wherein the recombinant TET enzyme catalyzes the oxidation of 5-methylcytosine (5mC) to 5-hydroxymethylcytosine (5hmC) and/or 5hmC to 5-formylcytosine (5fC) and/or 5-carboxycytosine (5caC).

60. The recombinant TET enzyme of claim 59, wherein the enzyme is modified by deletion of the TET LCR.

61. The recombinant TET enzyme of any one of claims 59 to 60, wherein the LCR is substituted with a heterologous linker sequence.

62. The recombinant TET enzyme of any one of claims 59 to 61, wherein the recombinant TET enzyme has an N-terminal truncation.

63. The recombinant TET enzyme of claim 62, wherein the N-terminal truncation extends to the N terminus of the catalytic domain of the recombinant TET enzyme.

64. The recombinant TET enzyme of any one of claims 59 to 63, wherein the enzyme comprises at least one purification tag selected from the group consisting of a his tag and a FLAG tag and combinations thereof.

65. A nucleic acid encoding the recombinant TET enzyme of any one of claims 59 to 64.

66. The nucleic acid of claim 65, further comprising a promoter in operable association with the nucleic acid encoding the recombinant TET enzyme.

67. A vector comprising the nucleic acid of any one of claims 65 to 66.

68. A host cell comprising the nucleic acid or vector of anyone of claims 65 to 67.

69. The host cell of claim 68, wherein the host cell is a prokaryotic host cell.

70. The host cell of claim 68, wherein the host cell is a eukaryotic host cell.

71. A method of modifying a nucleic acid in a nucleic acid sample comprising 5mC and/or 5hmC comprising:
contacting the nucleic acid with the engineered or recombinant TET enzyme of any one of claims 1 to 43 and 58 to 64 so that 5mC and/or 5hmC is converted 5fC and/or 5caC to provide an oxidized target nucleic acid.

72. The method of claim 71, wherein the engineered or recombinant TET enzyme is the engineered TET enzyme of any one of claims 1 to 43.

73. The method of any one of claims 71 to 72, further comprising the step of contacting the oxidized target nucleic acid with a reducing agent.

74. The method of claim 73, wherein the reducing agent is a borane reducing agent and the 5fC and/or 5caC is reduced to dihydrouracil (DHU).

75. The method of claim 74, wherein the borane reducing agent comprises an agent selected from the group consisting of 2-picoline borane (pic-BH3), borane, sodium borohydride, sodium cyanoborohydride, and sodium triacetoxyborohydride.

76. The method of any one of claims 71 to 72, further comprising contacting the oxidized target nucleic acid with sodium bisulfite so that the 5fC and/or 5caC is deaminated to uracil (U).

77. The method of any one of claims 71 to 72, further comprising contacting the oxidized target nucleic acid with an APOBEC (Apolipoprotein B mRNA Editing Catalytic Polypeptide-like) enzyme so that the 5fC and/or 5caC is deaminated to uracil (U).

78. The method of any one of claims 71 to 77, further comprising adding a blocking group to one or more of the 5mC and/or 5hmC in the nucleic acid sample.

79. The method of claim 78, wherein the blocking group is added prior to contacting with the oxidizing agent.

80. The method of any one of claims 78 to 79, wherein 5hmC is blocked.

81. The method of claim 80, wherein the blocking group comprises a sugar or a uridine diphosphate (UDP)-linked sugar.

82. The method of claim 78, wherein the blocking group is added after contacting with the oxidizing agent and prior to contacting with the borane reducing agent.

83. The method of claim 82, wherein the one or more modified cytosines comprises 5caC or 5fC.

84. The method of claim 83, wherein the blocking group comprises an aldehyde reactive compound.

85. The method of claim 84, wherein the aldehyde reactive compound comprises a hydroxylamine derivative, a hydrazine derivative, or a hydrazide derivative.

86. The method of claim 83, wherein adding the blocking group comprises contacting the nucleic acid sample with (i) a coupling agent and (ii) an amine, hydrazine, or hydroxylamine compound.

87. The method of any one of claims 71 to 86, further comprising sequencing the nucleic acid after contacting with the borane reducing agent to identify converted cytosine bases.

88. A system or kit for oxidizing a methylated nucleotide comprising:
the engineered or recombinant TET enzyme of any one of claims 1 to 43 and 58 to 64.

89. The system or kit of claim 88, further comprising a borane reducing agent.

90. The system of kit of claim 89, wherein the borane reducing agent comprises an agent selected from the group consisting of 2-picoline borane (pic-BH3), borane, sodium borohydride, sodium cyanoborohydride, and sodium triacetoxyborohydride.

91. The system of kit of claim 88, further comprising sodium bisulfite.

92. The system of kit of claim 88, further comprising an APOBEC (Apolipoprotein B mRNA Editing Catalytic Polypeptide-like) enzyme.

93. The system or kit of any one of claims 88 to 92, further comprising a blocking reagent.

94. The system or kit of claim 93, wherein the blocking reagent is selected from the group consisting of a sugar or a uridine diphosphate (UDP)-linked sugar and an aldehyde reactive compound.

95. The system or kit of claim 94, wherein the aldehyde reactive compound is selected from the group consisting of a hydroxylamine derivative, a hydrazine derivative, and a hydrazide derivative.

96. The system or kit of claim 94, wherein the blocking reagent is a sugar or a uridine diphosphate (UDP)-linked sugar and the system or kit further comprises a glucosyltransferase enzyme.


SEQUENCE LISTING (43 sequences)

SEQ ID NO: 1 - protein, 331 aa, Synthetic construct
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKYGIPTNRRCSQNETRNCTCQGENPETCGASFSFGCSWSMYYNGCKFARSKKPRKFRLHGAEPKEEERLGSHLQNLATVIAPIYKKLAPDAYNNQVEFEHQAPDCRLGLKEGRPFSGVTCCLDFCAHSHRDQQNMPNGSTVVVTLNREDNREVGACPEDEQFHVLPMYIIAPEDEFGSTEGQEKKIRMGSIEVLQSFRRRRVIRIG

SEQ ID NO: 2 - protein, 114 aa, Synthetic construct
VQEIEYWSDSEHNFQDPSIGGVAIAPTHGSILIECAKKEVHATTKVNDPDRNHPTRISLVLYRHKNLFLPKHGLALWEAKCACKARKEEECGKNGSDHVSQKNHGKQEKREPTG

SEQ ID NO: 3 - protein, 335 aa, Synthetic construct
EFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCTCQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTCCMDFCAHAHKDQHNLYNGCTVVCTLTKEDNRCVGQCPEDEQLHVLPLYKMASTDEFGSEENQNAKVSSGAIQVLTAFPREVRRLPEPAKS

SEQ ID NO: 4 - protein, 160 aa, Synthetic construct
EELWSDSEHNFLDESIGGVAVAPAHCSILIECARKELHATTPLKKPNRCHPTRISLVFYQHKNLNQPNHGLALWEAKCKQLACRARQRQEEAARLGLGQQEAKLYGKKRKWGGAMVAEPQHKEKKGAIPTRQALAMPTDSAVTVSSYAYTKVTGPYSRWI

SEQ ID NO: 5 - protein, 351 aa, Synthetic construct
EEDIKLSPEQIERLKANARGDVPGCECFPPDQLPPEPGPYYTHLGTAKSLEELRENVEKRTGLTGKAIRIEKVLFTGKEGKTTSGCPLGKWIIRRSGPEEKVLMVVKHRKGHVCVNSWIVIVTVAWEGVPAAMADDLYATVTDKLKNYGVPTSRRCGVNEPRTCTCQGLDNTTCGASFSFGCSWSMYYNGCKFARSQTARKFKLSVKTEEQCMEDKLQNLATYLSPVYKTMAPESFSNMTQHEQEAVDCRLGLQPGRPWSGVTCCFDFCAHAHRDIHNMNNGCTTVVTLTKHRGFECPEDEQLHVLPLYVIDQSDEFGSKEGYHEKVRSGAIEVLTKYPCEIRTRQAPLES

SEQ ID NO: 6 - protein, 198 aa, Synthetic construct
VTDFTNNEDCFSDPSVGGVAIALSHGAVLFEVAKKELHATTALKIPNRFHPTRISLVFYQHRNLNKSDHGRGEWKEKCRCKKELAEKHAEMAEMAERAAELAEKATSTASIATQSLDVQTEPQPVGAIMDHMPYNYYPMQQQPPSPLNRYSPMVATSSIQSSPSPQMQPVSTPSAWGNLFPLHPSVVQGPYQESETLG

SEQ ID NO: 7 - protein, 323 aa, Synthetic construct
EKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTPTNRRCALNEERTCTCQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTCCLDFCAHAHRDLHNMQNGSTLVCTLTREDNREFGGCPEDEQLHVLPLYKVSDVDEFGSVEAQEEKKRSGAIQVLSSFRRKVRMLAEPVKT

SEQ ID NO: 8 - protein, 159 aa, Synthetic construct
DEVWSDSEQSFLDPSIGGVAVAPTHGSILIECAKKELHATTPLKNPNRNHPTRISLVFYQHKSMNEPKHGLALWEAKCACKAREKEEECEKYGPDYVPQKSHGKKVKREPAEPHETSEPTYLRFIKSLAERTMSVTTDSTVTTSPYAFTRVTGPYNRYI

SEQ ID NO: 9 - protein, 1912 aa, Mus musculus
MEQDRTTHAEGTRLSPFLIAPPSPISHTEPLAVKLQNGSPLAERPHPEVNGDTKWQSSQSCYGISHMKGSQSSHESPHEDRGYSRCLQNGGIKRTVSEPSLSGLHPNKILKLDQKAKGESNIFEESQERNHGKSSRQPNVSGLSDNGEPVTSTTQESSGADAFPTRNYNGVEIQVLNEQEGEKGRSVTLLKNKIVLMPNGATVSAHSEENTRGELLEKTQCYPDCVSIAVQSTASHVNTPSSQAAIELSHEIPQPSLTSAQINFSQTSSLQLPPEPAAMVTKACDADNASKPAIVPGTCPFQKAEHQQKSALDIGPSRAENKTIQGSMELFAEEYYPSSDRNLQASHGSSEQYSKQKETNGAYFRQSSKFPKDSISPTTVTPPSQSLLAPRLVLQPPLEGKGALNDVALEEHHDYPNRSNRTLLREGKIDHQPKTSSSQSLNPSVHTPNPPLMLPEQHQNDCGSPSPEKSRKMSEYLMYYLPNHGHSGGLQEHSQYLMGHREQEIPKDANGKQTQGSVQAAPGWIELKAPNLHEALHQTKRKDISLHSVLHSQTGPVNQMSSKQSTGNVNMPGGFQRLPYLQKTAQPEQKAQMYQVQVNQGPSPGMGDQHLQFQKALYQECIPRTDPSSEAHPQAPSVPQYHFQQRVNPSSDKHLSQQATETQRLSGFLQHTPQTQASQTPASQNSNFPQICQQQQQQQLQRKNKEQMPQTFSHLQGSNDKQREGSCFGQIKVEESFCVGNQYSKSSNFQTHNNTQGGLEQVQNINKNFPYSKILTPNSSNLQILPSNDTHPACEREQALHPVGSKTSNLQNMQYFPNNVTPNQDVHRCFQEQAQKPQQASSLQGLKDRSQGESPAPPAEAAQQRYLVHNEAKALPVPEQGGSQTQTPPQKDTQKHAALRWLLLQKQEQQQTQQSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 10 - protein, 1803 aa, Mus musculus
MSQFQVPLAVQPDLSGLYDFPQGQVMVGGFQGPGLPMAGSETQLRGGGDGRKKRKRCGTCDPCRRLENCGSCTSCTNRRTHQICKLRKCEVLKKKAGLLKEVEINAREGTGPWAQGATVKTGSELSPVDGPVPGQMDSGPVYHGDSRQLSTSGAPVNGAREPAGPGLLGAAGPWRVDQKPDWEAASGPTHAARLEDAHDLVAFSAVAEAVSSYGALSTRLYETFNREMSREAGSNGRGPRPESCSEGSEDLDTLQTALALARHGMKPPNCTCDGPECPDFLEWLEGKIKSMAMEGGQGRPRLPGALPPSEAGLPAPSTRPPLLSSEVPQVPPLEGLPLSQSALSIAKEKNISLQTAIAIEALTQLSSALPQPSHSTSQASCPLPEALSPSAPFRSPQSYLRAPSWPVVPPEEHPSFAPDSPAFPPATPRPEFSEAWGTDTPPATPRNSWPVPRPSPDPMAELEQLLGSASDYIQSVFKRPEALPTKPKVKVEAPSSSPAPVPSPISQREAPLLSSEPDTHQKAQTALQQHLHHKRNLFLEQAQDASFPTSTEPQAPGWWAPPGSPAPRPPDKPPKEKKKKPPTPAGGPVGAEKTTPGIKTSVRKPIQIKKSRSRDMQPLFLPVRQIVLEGLKPQASEGQAPLPAQLSVPPPASQGAASQSCATPLTPEPSLALFAPSPSGDSLLPPTQEMRSPSPMVALQSGSTGGPLPPADDKLEELIRQFEAEFGDSFGLPGPPSVPIQEPENQSTCLPAPESPFATRSPKKIKIESSGAVTVLSTTCFHSEEGGQEATPTKAENPLTPTLSGFLESPLKYLDTPTKSLLDTPAKKAQSEFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTACMDFCAHAHKDQHNLYNGCTVVCTLTKEDNRCVGQIPEDEQLHVLPLYKMASTDEFGSEENQNAKVSSGAIQVLTAFPREVRRLPEPAKSCRQRQLEARKAAAEKKKLQKEKLSTPEKIKQEALELAGVTTDPGLSLKGGLSQQSLKPSLKVEPQNHFSSFKYSGNAVVESYSVLGSCRPSDPYSMSSVYSYHSRYAQPGLASVNGFHSKYTLPSFGYYGFPSSNPVFPSQFLGPSAWGHGGSGGSFEKKPDLHALHNSLNPAYGGAEFAELPGQAVATDNHHPIPHHQQPAYPGPKEYLLPKVPQLHPASRDPSPFAQSSSCYNRSIKQEPIDPLTQAESIPRDSAKMSRTPLPEASQNGGPSHLWGQYSGGPSMSPKRTNSVGGNWGVFPPGESPTIVPDKLNSFGASCLTPSHFPESQWGLFTGEGQQSAPHAGARLRGKPWSPCKFGNGTSALTGPSLTEKPWGMGTGDFNPALKGGPGFQDKLWNPVKVEEGRIPTPGANPLDKAWQAFGMPLSSNEKLFGALKSEEKLWDPFSLEEGTAEEPPSKGVVKEEKSGPTVEEDEEELWSDSEHNFLDENIGGVAVAPAHCSILIECARRELHATTPLKKPNRCHPTRISLVFYQHKNLNQPNHGLALWEAKMKQLAERARQRQEEAARLGLGQQEAKLYGKKRKWGGAMVAEPQHKEKKGAIPTRQALAMPTDSAVTVSSYAYTKVTGPYSRWI

SEQ ID NO: 11 - protein, 1520 aa, Synthetic construct
QQHQQQQHQQQQQQMQEAQHQRYQQQMNQAQMQHSQKSPQMIKDDQQQMRHQQPSPQMHQPQTSTQSQHLRMQQQSPMMSHNQMQQQVQMHQQQAQSQQQAQMHQAQSQSQQQEQMHQQQLAQLRQQNQLHHQNVRQPLQIQPQQSHVSLHHQQLPSPVQQHQHIQQNQMTHHNMQGNQGSQSLHMMQHQSNVHHHHQQMMGGQGSSPQHQEMWSGQSNSRPPSQNPPEPWPNESANQQQMWTNDKKSNQFNERAELNNRLKTMILNKQQQEIKKIQDGGHMGYQDQKNLPPVSSGNFLALGHHPRKLVSEGGGYRSGGSYRNDGLHAMENIVNSVKCSDDFVYSNTPVSQPGSFLQRMASSKYYQVTSQTQTSSQTLSKVSSPVPQPSMRSPSPLGVQKIKQEPQWDEKPWGTSIDPGRSSHQPVQDQVVKTEMDAGCENPGTPFGEDRLKAVKEESFDSDPSKEYSFEDVDIKTEEDIKLSPEQIERLKANARGDVPGCECFPPDQLPPEPGPYYTHLGTAKSLEELRENVEKRTGLTGKAIRIEKVLFTGKEGKTTSGCPLGKWIIRRSGPEEKVLMVVKHRKGHVCVNSWIVIVTVAWEGVPAAMADDLYATVTDKLKNYGVVTSRRCGVNEPRTCACQGLDNTTCGASFSFGCSWSMYYNGCKFARSQTARKFKLSVKTEEQCMEDKLQNLATYLSPVYKTMAPESFSNMTQHEQEAVDCRLGLQPGRPWSGVTACFDFCAHAHRDIHNMNNGCTTVVTLTKHRGFEKPEDEQLHVLPLYVIDQSDEFGSKEGYHEKVRSGAIEVLTKYPCEIRTRQAPLESCRKLAKKRKDEREHAKRALAYKMSMNLDYSSASPQPQPSNFNDHPVQNGLQSEVQHNYLSYPTTPEKGSKSRGMSLHHQASEMESMSQMVQSPHRPKVYPPSPFRSPLRVVTQSPGSPYSAPTTPEMGKADRDAYYNPASSPTSVFRRPVGRPPSRSSNPATPQPHMDSTYLKPHRMGSPYSQGMESDRNFYQSIPSHSPIQQPYSPVYSREHSYTLPSINSFISPLRDGFGSKNTAFNTYSSRSYESQSAPVSPVYSTANLQRPISTYSAGSPGMMRASSPGSKCSSVSTQCSPSRSPYPTTLQAPSPNPSQSSGYWSGSQSDVRETFYPRDHPGVPFSPPKWSPLSRHSRSNLSPSQATFSTPTTPTYVSTQQSLPSFQNFTRKTVDQTDFAWNRYSPVPAMSQDHNWRPVEPNIQKNYMSSQNFSGLPSYQIEYPYEKKTAVQSENTYNVTEPSVSPSKQWARESQTPPPWSGSNQSLPRTPDVEPQNLLGSVTDFTNNEDCFSDPNVGGVAIALSHGAVLFEVAKHELHATTALKIPNRFHPTRISLVFYQHRNLNKSDHGRGEWKEKQRCKKELAEKHAEMAEMAERAAELAEKATSTASIATQSLDVQTEPQPVGAIMDHMPYNYYPMQQQPPSPLNRYSPMVATSSIQSSPSPQMQPVSTPSAWGNLFPLHPSVVQGPYQESETLG

SEQ ID NO: 12 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 13 - protein, 15 aa, Synthetic construct
GGGGSGGGGSGGGGS

SEQ ID NO: 14 - protein, 8 aa, Synthetic construct
DYKDDDDK

SEQ ID NO: 15 - protein, 460 aa, Synthetic construct
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKYGIPTNRRCSQNETRNCTCQGENPETCGASFSFGCSWSMYYNGCKFARSKKPRKFRLHGAEPKEEERLGSHLQNLATVIAPIYKKLAPDAYNNQVEFEHQAPDCRLGLKEGRPFSGVTCCLDFCAHSHRDQQNMPNGSTVVVTLNREDNREVGACPEDEQFHVLPMYIIAPEDEFGSTEGQEKKIRMGSIEVLQSFRRRRVIRIGGGGGSGGGGSGGGGSVQEIEYWSDSEHNFQDPSIGGVAIAPTHGSILIECAKKEVHATTKVNDPDRNHPTRISLVLYRHKNLFLPKHGLALWEAKCACKARKEEECGKNGSDHVSQKNHGKQEKREPTG

SEQ ID NO: 16 - protein, 479 aa, Synthetic construct
MGHHHHHHDYKDDDDKGGSQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKYGIPTNRRCSQNETRNCTCQGENPETCGASFSFGCSWSMYYNGCKFARSKKPRKFRLHGAEPKEEERLGSHLQNLATVIAPIYKKLAPDAYNNQVEFEHQAPDCRLGLKEGRPFSGVTCCLDFCAHSHRDQQNMPNGSTVVVTLNREDNREVGACPEDEQFHVLPMYIIAPEDEFGSTEGQEKKIRMGSIEVLQSFRRRRVIRIGGGGGSGGGGSGGGGSVQEIEYWSDSEHNFQDPSIGGVAIAPTHGSILIECAKKEVHATTKVNDPDRNHPTRISLVLYRHKNLFLPKHGLALWEAKCACKARKEEECGKNGSDHVSQKNHGKQEKREPTG

SEQ ID NO: 17 - protein, 972 aa, Synthetic construct
EFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCTCQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTCCMDFCAHAHKDQHNLYNGCTVVCTLTKEDNRCVGQCPEDEQLHVLPLYKMASTDEFGSEENQNAKVSSGAIQVLTAFPREVRRLPEPAKSCRQRQLEARKAAAEKKKLQKEKLSTPEKIKQEALELAGVTTDPGLSLKGGLSQQSLKPSLKVEPQNHFSSFKYSGNAVVESYSVLGSCRPSDPYSMSSVYSYHSRYAQPGLASVNGFHSKYTLPSFGYYGFPSSNPVFPSQFLGPSAWGHGGSGGSFEKKPDLHALHNSLNPAYGGAEFAELPGQAVATDNHHPIPHHQQPAYPGPKEYLLPKVPQLHPASRDPSPFAQSSSCYNRSIKQEPIDPLTQAESIPRDSAKMSRTPLPEASQNGGPSHLWGQYSGGPSMSPKRTNSVGGNWGVFPPGESPTIVPDKLNSFGASCLTPSHFPESQWGLFTGEGQQSAPHAGARLRGKPWSPCKFGNGTSALTGPSLTEKPWGMGTGDFNPALKGGPGFQDKLWNPVKVEEGRIPTPGANPLDKAWQAFGMPLSSNEKLFGALKSEEKLWDPFSLEEGTAEEPPSKGVVKEEKSGPTVEEDEEELWSDSEHNFLDESIGGVAVAPAHCSILIECARKELHATTPLKKPNRCHPTRISLVFYQHKNLNQPNHGLALWEAKCKQLACRARQRQEEAARLGLGQQEAKLYGKKRKWGGAMVAEPQHKEKKGAIPTRQALAMPTDSAVTVSSYAYTKVTGPYSRWI

SEQ ID NO: 18 - protein, 862 aa, Synthetic construct
EKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTPTNRRCALNEERTCTCQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTCCLDFCAHAHRDLHNMQNGSTLVCTLTREDNREFGGCPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPSIGGVAVAPTHGSILIECAKKELHATTPLKNPNRNHPTRISLVFYQHKSMNEPKHGLALWEAKCACKAREKEEECEKYGPDYVPQKSHGKKVKREPAEPHETSEPTYLRFIKSLAERTMSVTTDSTVTTSPYAFTRVTGPYNRYI

SEQ ID NO: 19 - protein, 1044 aa, Synthetic construct
EEDIKLSPEQIERLKANARGDVPGCECFPPDQLPPEPGPYYTHLGTAKSLEELRENVEKRTGLTGKAIRIEKVLFTGKEGKTTSGCPLGKWIIRRSGPEEKVLMVVKHRKGHVCVNSWIVIVTVAWEGVPAAMADDLYATVTDKLKNYGVPTSRRCGVNEPRTCTCQGLDNTTCGASFSFGCSWSMYYNGCKFARSQTARKFKLSVKTEEQCMEDKLQNLATYLSPVYKTMAPESFSNMTQHEQEAVDCRLGLQPGRPWSGVTCCFDFCAHAHRDIHNMNNGCTTVVTLTKHRGFECPEDEQLHVLPLYVIDQSDEFGSKEGYHEKVRSGAIEVLTKYPCEIRTRQAPLESCRKLAKKRKDEREHAKRALAYKMSMNLDYSSASPQPQPSNFNDHPVQNGLQSEVQHNYLSYPTTPEKGSKSRGMSLHHQASEMESMSQMVQSPHRPKVYPPSPFRSPLRVVTQSPGSPYSAPTTPEMGKADRDAYYNPASSPTSVFRRPVGRPPSRSSNPATPQPHMDSTYLKPHRMGSPYSQGMESDRNFYQSIPSHSPIQQPYSPVYSREHSYTLPSINSFISPLRDGFGSKNTAFNTYSSRSYESQSAPVSPVYSTANLQRPISTYSAGSPGMMRASSPGSKCSSVSTQCSPSRSPYPTTLQAPSPNPSQSSGYWSGSQSDVRETFYPRDHPGVPFSPPKWSPLSRHSRSNLSPSQATFSTPTTPTYVSTQQSLPSFQNFTRKTVDQTDFAWNRYSPVPAMSQDHNWRPVEPNIQKNYMSSQNFSGLPSYQIEYPYEKKTAVQSENTYNVTEPSVSPSKQWARESQTPPPWSGSNQSLPRTPDVEPQNLLGSVTDFTNNEDCFSDPSVGGVAIALSHGAVLFEVAKKELHATTALKIPNRFHPTRISLVFYQHRNLNKSDHGRGEWKEKCRCKKELAEKHAEMAEMAERAAELAEKATSTASIATQSLDVQTEPQPVGAIMDHMPYNYYPMQQQPPSPLNRYSPMVATSSIQSSPSPQMQPVSTPSAWGNLFPLHPSVVQGPYQESETLG

SEQ ID NO: 20 - protein, 481 aa, Synthetic construct
EAPDCDCFTSSDTKAPSEPGSYYTHLGAASSLEELRRETETRVGLSGKQLRIEKVVYTGKEGKSSQGCPIAKWVIRRVDPEEKLLFIVKRRQGHRCKAAFIVICIVVWDGIPTQEADSVYRMLSVKLNKYGLPTVRRCATNENRTCACQGLDPETCGVSYSFGCSWSMYYNGCKYARSKTVRKFRLSVKNEEAEIEERMNILATMLSPLYVTVAPQAFQNQVQYEREAPDCRLGLKPGKPFSGVTCCLDFCAHTHRDLHNMQDGCTVQVTLLKPLPPGVKADDEQLHILPLYTMDTTDEFDSEEGQKKKAETGAVQVLEKFATEVRVRSTPLQGGGGSGGGGSGGGGSEVTETNENVDCFEDPQMGGVAIALPHGSVVIECAKLEMHSTTALKKPNRLNPTRMTLIFYQHRNLNRPKHGIAEWAEKMRLKKLGLATNDPIDDKDMLLMQEDDIKSEPLDDLHHMRHMGDEHDMMDVEYSGV

SEQ ID NO: 21 - protein, 479 aa, Synthetic construct
EAPDCDCFSSGTDKAPSEPGSYYTHLGCAATLEDLRRETELRVGLTGKQLRIEKVVYTGKEGKSSQGCPIAKWVIRRVDPEEKLLFVVKRRQGHRCKASFIVICIVVWDGIPTHEADSVYRMLAVKLNKFGLPTVRRCATNENRTCACQGLDPELCGVSYSFGCSWSMYYNGCKYARSKTVRKFRLSVKNEEAEIEERMNVLATMLSPLYVTVAPQAFQNQVQYEREAPDCRLGLKPGKPFSGVTCCLDFCAHTHRDLHNMQDGCTVQVTLLKPLPPGVKPDDEQLHVLPLYTMDTTDEFDSEEGQKKKHETGAVQVLEKFQTEVRVRSTPLQGGGGSGGGGSGGGGSEVTETNENLDCFEDPQMGGVAIALPHGSVIIECAKLEMHSTTAVKKPNRLNPTRMTLIFYQHRNLNRPKHGIAEWAEKMRLKKLGIPTNDPLDDKDMLLEDDIKAEPLDELHHMRHIGDEHDMMDVEYPGV

SEQ ID NO: 22 - protein, 608 aa, Synthetic construct
EIPECECFQSDKNPPEPGTYYTHLGTASSLMELRREFEERCNLTGRQLRIEKIVYTGKEGKTTQGCPVAKWVIRRADMEEKILVVVKKRPGHRCIAAYIVVCMVAWDGMPRLEADNAYKNLIPKLNKYGLPTTRRCATNENRTCACQGLDPETSGASYSFGCSWSMYYNGCKYARSKTVRKFRLSVKSEEAAIEDHMNLIATLLAPVFKQVCPRSYDNQTKYEQEASDCRLGLEPGKPFSGVTACLDFCAHSHRDLHNMQDGCTVHVALLKPGNRDTRLPDDEQFHVLPLYTMDGTDEFESVEGQRDKHRTGAVQMLDKFPCEVRVRSTPLIGGGGSGGGGSGGGGSEVAEINENIEAFQDPQMGGVAIALNHGSVLIECAKHEMHATTAVRRPDRHHPTRMTLIFYQHRNLNRSRHGIDEWEEKMRVKKINTDLDNKAKEERERLIKKAAGEDLDELDEDALMDDAPVPVVKKETNGQALKNGASGSKKKKNNSTAESKKSQSGEQSKNEKVALHAPTLTTTSWTTLFPMHPCVVTGTYPEGNSSPTSSTNNNSNSNAGNGQSLPPGSGPQVPPGQPGGAAAIPQQQHPHPHPQLQQQQHQPQQQPT

SEQ ID NO: 23 - protein, 604 aa, Synthetic construct
EIPECECFQSDKNPPEPGTYYTHLGTASSLMDLRREFEERCNLTGRQLRIEKIVYTGKEGKTSQGCPVAKWVIRRADLEEKILVVVKKRPGHRCIAAYIVVCMVAWDGMPRLEADNAYKNLIPKLNKYGLPTTRRCATNENRTCACQGLDPESSGASYSFGCSWSMYYNGCKYARSKTVRKFRLSVKSEEAAIEDHMNLIATLLAPVFKQVCPRSYDNQTKYEHEASDCRLGLEPGKPFSGVTACLDFCAHSHRDLHNMQDGCTVHVALLKPGNRDTRLPDDEQFHVLPLYTMDGTDEFESVEGQRDKHRTGAVQMLDKFPCEVRVRSTPLIGGGGSGGGGSGGGGSEVAEINENIEAFQDPQMGGVAIALNHGSVLIECAKHEMHATTAVRRPDRHHPTRMTLIFYQHRNLNRCRHGIDEWEEKMRVKKINTDLDNKAKEERERLIKKAAGEDMDELDEDALMQDEPVPIKKESSANGQQLKNGASGSKKKKSSDSKKSQANEQSKNEKVALHAPTLTTTSWTTLFPTHPCVVSGKYPEGNSSPTSSTNNAPNGGGCPAQQQQHLPPPPGSGLIHPPPGTPTGTAAPPPLPTPHQQLPQQQQT

SEQ ID NO: 24 - protein, 495 aa, Synthetic construct
EVPSCDCPDRGIQKDKGPYYTHLGAGPSVAAVREIMETRCGHKGKAVRIEKLVYTGKEGKSSQGCPVAKKVIRRSSEEEEVLCLVRERPGHQCQTAVMVMLIVVWDGIPRPMADRLYTELTESLKSYNGHPTDRRCTLNENRTCTCQGTDPETCGASFSFGCSWSMYFNGCKFGRSPSPRRFRIDPSSPLNEKNLEDNLQNLATELAPIYKQYAPVAYQNQVEYEHVARECRLGRKEGRPFSGVTACLDFCAHPHRDIHNMNNGSTVVCTLTREDNRSLGVVPEDEQLHVLPLYKLSDTDEFGSKEGMEAKIRSGAVQVLQPTRRKRTRFTQPVGGGGSGGGGSGGGGSDEYWSDNEHVFLDDSVGGVAIAPTHGSVLIECARRELHATTPVANPNRNHPTRLSLVLYQHKNLNKPQHGFELNKIKFEAKEAKNKKTKASEQKDRSAEEAAALFPEVNELNQIPSHKALTLTHDNVVTVSPYALTHVAGPYNHWV

SEQ ID NO: 25 - protein, 492 aa, Synthetic construct
LPNCNCLDRVIQKDKGPYYTHLGAGPSVAAVREIMENRYGQKGNAIRIEIVEYTGKKTSHGCPIAKWVLRRSSDEEKVLCLVRQRTGHHCPTAVMVVLIMVWDGIPLPMADRLYSELTGNLKSYNGHPTDRRCTLNESRTCTCQGTDPETCGASFSFGCSWSMYFNGCKFGRSPSPRRFRIDPSSPLHEKNLEDNLQSLATELAPIYKQYAPVAYHNQVAYEHVARECRLGSKDGRPFSGVTACLDFCAHPHRDIHNMNNGSTVVCTLTREDNRSLGFIPQDEQLHVLPLYKLSDTDEFGSREGMEAKIKSGAIEVLTPRPKKRRRFTQPVGGGGSGGGGSGGGGSEEYWSDSEHIFLDANIGGVAIAPSHGSVLIECARRELHATTPVEHPNRNHPTRLSLVFYQHKNLNKPQHGFELNKIKFEAKEAKNKKTKASEQKDQAANEGPELFPEVNEFNQIPSHKALTLTHDNVVTVSPYALTHVAGPYNHWV

SEQ ID NO: 26 - protein, 509 aa, Synthetic construct
EFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMASTDEFGSEENQNAKVGSGAIQVLTAFPREVRRLPEPAGGGGSGGGGSGGGGSEEEEEELWSDSEHNFLDENIGGVAVAPAHGSILIECARRELHATTPLKKPNRCHPTRISLVFYQHKNLNQPNHGLALWEAKMKQLAEARQEEAARLGLGQQEAKLYGKKRKWGGAMVAEPQQKEKKGVVPTRQALAVPTDSAVTVSSYAYTKVTGPYSRWI

SEQ ID NO: 27 - protein, 517 aa, Synthetic construct
ESDTPGVQNQNNGCQGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGVCTNRRCSQNETKRMPSPPRNCCCQGENPETCGASFSFGCSWSMYYNGCKFARSKKPRKFRLHGDEPKEEERLGSHLQNLATVIAPIYKKLAPDAYSNQVEFEHQAPDCCLGLKEGRPFSGVTACLDFSAHAHRDQQNMPNGSTVVVTLNREDNREVGAKPEDEQFHVLPLYIIAPEDEFGSMEGQEEKIRTGSIEVLQSFRRKRVIRIGGGGSGGGGSGGGGSVKEIEYWSDSEHNFQDPCIGGVAIAPTHGSILIECAKCEVHATTKVNDPDRNHPTRISLVLYRHKNLFLPKHCLALWEAKMAEKARKEKECGKNGSDHVSRKNHGKQVKREPTGPQEPSYLRFIQSLAENTGSVTTDSTVTTSPYAFTQVTGPYNTFV

SEQ ID NO: 28 - protein, 507 aa, Synthetic construct
FPSCRCVEQIIEKDEGPFYTHLGAGPNVAAIREIMEERFGQKGKAIRIEKVIYTGKEGKSSQGCPIAKWVIRRSCSEEKLLCLVRERAGHTCEAAVIVILILLWEGIPQSLATELYSELTETLKNHGTLTNRRCALNEERTCACQGLDPETCGASFSFGCSWSMYYNGCKFARSKVPRKFKLLGDDPKEEEKLESHLQNLSTLLAPIYKKLAPDAYNNQIEYEHRAPECRLGLKEGRPFSGVTACLDFCAHAHRDLHNMHNGSTVVCTLTKEDNREIGAKPDDEQLHVLPLYKISDVDEFGSVEAQEEKKRNGAIEVLTSFRRKVRMLAGGGGGSGGGGSGGGGSGENDEVWSDSEQNFLDPDIGGVAVAPAHGSVLIECAKRELHATTPLTDPNRNHPSRVSLVFYQHKSMNEPKHGLALWEAKMAEKAREKEEECEKYGPDYVPQKTHGKKGKREPTEPQEPSERTYLRFFRSLAERTMSATTDSTITTSPYAFTRVTGPYNRYI

SEQ ID NO: 29 - protein, 528 aa, Synthetic construct
LSGFLESPLKYLTSPTKSLIDTPAKMAQAEFPTCDCVEQINEKDEGPYYTHLGSGPTVASIRELMEERFGEKGEAIRIEKVIYTGKEGKSSRGCPIAKWVIRRQSEDEKLMCLVRQRAGHHCENAVIIILIMAWEGIPRALGDSLYDDISGTITKYGNPTSRRCGLNDDRTCACQGKDPNTCGASFSFGCSWSMYFNGCKYARSKTPRKFRLIGDNPKEEEFLKDSFQDLATKVAPVYKMLAPQAYQNQANNEDVAIDCRLGLEEGRPFSGVTACMDFCAHAHKDQHNLYNGCTVVCTLTKEDNRMIGKIAEDEQLHVLPLYKVSTTDEFGSEERQLEKIRKGGIQVLSSFPREVRKLSEPAGGGGSGGGGSGGGGSDEPLDGNIGGVAVAPGHGSILIECARRELHATTPLKKPNRCHPTRISLVFYQHKNLNQPNHGLALWEAKMKQLAERARVKEEEAAKLGIKQEVKSLGKKRKWGGAATTETPPVEKKDFIPTRQAATSLTDSTTTAFSYAYTKVTGPYSRFI

SEQ ID NO: 30 - protein, 513 aa, Synthetic construct
EFPTCDCVEHILEKDEGPYYNHLGSGPTVASIRELMEERFGEKGEAIRIEKVVFTGREGKSSQGCPIAKWVIRRGSETEKLLCLVRHRAGHHCANAVIIVVILAWEGVPRALGDKLYHELSDILTKYGNPTSRRCGLNEDRTCACQGKDPETCGASFSFGCSWSMYFNGCKYARSKTPRKFRLQGDHPKEEENLRDNFQNLATHVAPLYKRLAPQAYSNQCLNEKVASDCRLGLKEGRPFSGVTACMDFCAHAHKDQHNLYNGCTVVCTLTKEDNRVVGQIPEDEQLHVLPLYKISTTDEFGSEENQQRKMETGAIQVLNSFRREVRKLPEPAGGGGSGGGGSGGGGSPEDEEVWSDSEHNFLDPNIGGVAVAPAHGSVLIECARRELHATTPLKKPNRCHPSRISLVFYQHKNLSQPCHGLALWEAKMKLLAERARQRQQEAALLGLPQDDIKAYGKKRKWASSAASPGPGQCKDKREGVVTRLAPTQHTTTMVTVSPFAFTQLTGHYSRFL

SEQ ID NO: 31 - protein, 552 aa, Synthetic construct
EFPTCDCVEQIVEKDEGPYYTHLGSGPTVASIRELMEERYGEKGKAIRIEKVIYTGKEGRAHRPRPVSLQVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFSGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALEMAGGGGGSGGGGSGGGGSEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGAMVTEPQHKEKKGLVPTRQALAMPTDSAVTVSSYAYTKVTGPYSRWI

SEQ ID NO: 32 - protein, 526 aa, Synthetic construct
ESPMKLLDTPIKNLLDTPLKTQYDIPSCHCVDQISERDEGPYYTHLGSAPTVAGIRKIMEERSGLTGSAIRIEKVVYTGKEGKSGQGCPIAKWVIRRANVDEKLLVLVRERAGHSCETSCIVVVILIWEGIQINLADRLYSELSDTLTKHGALTNRRCALNEERTCACQGFEADACGASFSFGCSWSMYYNGCKFARSKIPRKFKLLGDDPKEEEKLEQNLQGLATLIAPTYKKIAPDAYTNQVEHEHRAPDCRLGLKEGRPFSGVTACLDFCAHAHRDLHNMQGGSTVVCTLTREDNREIGKIPEDEQLHVLPLYKASSTDEFGSAEAQLEKTKTGAIQVLSAFRRQVRMLGGGGSGGGGSGGGGSKEKEDVWSDSEHNFLDPEIGGVAVAPSHGSIIIECAKRELHATTPLKKPDRNHPTRISLVFYQHKNLNEAKHGLALWEAKMAEKAREKEEDAEKYGTENTSNKSSGKKVKREHSEPSEPPYKQFLLMLNERSTSCTTNTYVSTSPYAFTKVTGPYNQFL

SEQ ID NO: 33 - protein, 548 aa, Synthetic construct
EFPTCDCVEQINEKDEGPYYTHLGSGPTVASIRELMEERFGQKGDAIRIEKVIYTGKEGKSSRGCPIAKWVIRRQSEDEKLMCLVRQRAGHHCENAVIIILIMAWEGIPRSLGDSLYNDITETITKYGNPTSRRCGLNDDRTCACQGKDPNTCGASFSFGCSWSMYFNGCKYARSKTPRKFRLIGENPKEEDGLKDNFQNLATKVAPVYKMLAPQAYQNQVNNEDIAIDCRLGLKEGRPFSGVTACMDFCAHAHKDQHNLYNGCTVVCTLTKEDNRMIGRVAEDEQLHVLPLYKVSTTDEFGSEEGQLEKIKKGGIHVLSSFPREVRKLSEPAKSCRQRQLEAKKAAAEKKKLQKEKLVSPDKTKQEPSDGGGGSGGGGSGGGGSEEDDEEIWSDSEHNFLDENIGGVAVAPGHGSILIECARRELHATTPLKKPNRCHPTRISLVFYQHKNLNQPNHGLALWEAKMKQLAERARAREEEAAKLGIKQEVKSLGKKRKWGGAATTETPPVEKKDYTPTRQAATILTDSATTSFSYAYTKVTGPYSRFI

SEQ ID NO: 34 - protein, 596 aa, Synthetic construct
EEDIKLSPEQIERLKANARGDVPGCECFPPDQLPPEPGPYYTHLGTAKSLEELRENVEKRTGLTGKAIRIEKVLFTGKEGKTTSGCPLGKWIIRRSGPEEKVLMVVKHRKGHVCVNSWIVIVTVAWEGVPAAMADDLYATVTDKLKNYGVVTSRRCGVNEPRTCACQGLDNTTCGASFSFGCSWSMYYNGCKFARSQTARKFKLSVKTEEQCMEDKLQNLATYLSPVYKTMAPESFSNMTQHEQEAVDCRLGLQPGRPWSGVTACFDFCAHAHRDIHNMNNGCTTVVTLTKHRGFEKPEDEQLHVLPLYVIDQSDEFGSKEGYHEKVRSGAIEVLTKYPCEIRTRGGGGSGGGGSGGGGSVSPSKQWARESQTPPPWSGSNQSLPRTPDVEPQNLLGSVTDFTNNEDCFSDPNVGGVAIALSHGAVLFEVAKHELHATTALKIPNRFHPTRISLVFYQHRNLNKSDHGRGEWKEKQRCKKELAEKHAEMAEMAERAAELAEKATSTASIATQSLDVQTEPQPVGAIMDHMPYNYYPMQQQPPSPLNRYSPMVATSSIQSSPSPQMQPVSTPSAWGNLFPLHPSVVQGPYQESETLG

SEQ ID NO: 35 - protein, 14 aa, Synthetic construct
HHHHHHDYKDDDDK

SEQ ID NO: 36 - protein, 24 aa, Synthetic construct
VIRIGGGGGSGGGGSGGGGSVQEI

SEQ ID NO: 37 - protein, 6 aa, Synthetic construct
PVPRCG

SEQ ID NO: 38 - protein, 6 aa, Synthetic construct
DEFWSD

SEQ ID NO: 39 - protein, 14 aa, Synthetic construct
KRAKMKQNHNKSGS

SEQ ID NO: 40 - protein, 13 aa, Synthetic construct
GGSGGGGSGGGGS

SEQ ID NO: 41 - protein, 20 aa, Synthetic construct
WEAKMAEKAREKEEECEKYG

SEQ ID NO: 42 - protein, 65 aa, Synthetic construct
NKIKCKCKKVTKKKPADRECPDVSPEANLSHQIPSRVASTLTRDNVVTVSPYSLTHVAGPYNRWV

SEQ ID NO: 43 - protein, 82 aa, Synthetic construct
WEAKMAEKARKEEECGKNGSDHVSQKNHGKQEKREPTGPQEPSYLRFIQSLAENTGSVTTDSTVTTSPYAFTQVTGPYNTFV